(19) United States
(12) Patent Application Publication
(10) Pub. No.: US 2024/0382648 A1
(43) Pub. Date: Nov. 21, 2024

(54) POLYACRYLAMIDE HYDROGEL FOR USE IN THE TREATMENT AND/OR PREVENTION OF OSTEOARTHRITIS

(71) Applicant: CONTURA INTERNATIONAL A/S, Søborg (DK)

(72) Inventor: Ieva ANKORINA-STARK, Nivå (DK)

(21) Appl. No.: 18/693,579

(22) PCT Filed: Sep. 20, 2022
(86) PCT No.: PCT/EP2022/076016

§ 371 Date: Mar. 20, 2024

(30) Foreign Application Priority Data
Sep. 20, 2021 (EP) 21197730.1

(51) Int. Cl. A61L 27/16; A61L 27/52; C08F 220/56

(52) U.S. Cl. CPC A61L 27/16; A61L 27/52; C08F 220/56; A61L 2400/06; A61L 2430/24

(57) ABSTRACT

The present invention relates to a polyacrylamide hydrogel for use in prevention and/or treatment of osteoarthritis for at least 26 weeks in humans diagnosed with KL grade 2 and/or KL grade 3 osteoarthritis. Thus, a long term prevention and/or treatment of osteoarthritis patient sub-groups is achieved.

### TECHNICAL FIELD OF THE INVENTION

The present invention relates to a polyacrylamide hydrogel for use in prevention and/or treatment of osteoarthritis for at least 26 weeks in a human diagnosed with KL grade 2 and/or KL grade 3 osteoarthritis.

### BACKGROUND OF THE INVENTION

Osteoarthritis (OA) is a painful, debilitating joint disease with no known cure. It is characterized by heat, pain, swelling, crepitus (a crackling, crinkly, or grating feeling or sound under the skin), and a decreased range of motion in affected joints. In humans it affects the hands, knees, hips, spine and other joints.

The common symptoms for all types of OA in humans include varied levels of pain. Pain symptoms can be treated with anti-inflammatory drugs such as NSAIDs. Another possibility is injection of steroids directly into the affected joint. Specific treatment depends on the underlying cause of the OA. Common to the various types of existing treatment is that they all have their disadvantages, e.g. short term treatment, toxicity and side-effects.

Visco-supplementation is the process of injecting a gel-like substance into the joint. The substance is thought of as an additive to the joint fluid, thus lubricating the cartilage, and improving joint flexibility. This method of treatment, however, requires ongoing injections, as benefits are only temporary, because the currently used substances are degradable within weeks to months. Substances used in visco-supplementation include hyaluronic acid, or HA (Legend®, Hylartin® and Synacid®, Synvisc, Synvisc-One, Euflexxa, Supartz etc) and poly-sulfated glycosaminoglycans (PSGAGS) such as Adequan®.

WO 02/16453 discloses the use of a polyacrylamide hydrogel (PAAG) for treating e.g. arthritis, where the treatment is considered to be based on a lubricating and cushioning effect of the hydrogel. WO 2012/123385 discloses use of PAAG in the treatment and/or prevention of joint swelling and/or bone oedema in a mammal suffering from arthritis. Neither WO 02/16453 nor WO 2012/123385 discloses prevention and/or treatment of osteoarthritis KL grade 2 and/or 3 in a mammal. Abstracts/Osteoarthritis and Cartilage 29 (2021) S10-S432” discloses some results from a clinical study (ref. no: H-19031685 under the Danish Health authorities and registered at (NCT04179552). The abstract discloses 6 ml intraarticular (IA) injection with PAAG in KL grade 2-4 knee OA patients and observes the results after 4, 12, 26 and 52 weeks. However, the abstract does not disclose prevention and/or treatment of osteoarthritis for the specific subgroups of KL grade 2 and/or 3 in a mammals. Also, long term treatment (104 weeks) is not disclosed in the abstract, and an at least 50% reduction in WOMAC pain subscale is also not disclosed in the abstract.

Common to the various types of existing treatment is that they all have their disadvantages, e.g. short term treatment, toxicity and side-effects.

Hence, there is a need for an alternative or improved way to prevent and/or treat OA in humans, preferably a long term treatment. Also, there is a need for an alternative or improved way to prevent and/or treat the pain associated with OA in a mammal which is also, preferably a long term treatment.

### SUMMARY OF THE INVENTION

Thus, an object of the present invention relates to a polyacrylamide hydrogel for use in prevention and/or treatment of osteoarthritis for at least 26 weeks, preferably for at least 52 weeks in a human diagnosed with KL grade 2 and/or KL grade 3 osteoarthritis.

Thus, an object of the present invention relates to a polyacrylamide hydrogel for use in prevention and/or treatment of osteoarthritis for at least 26 weeks, preferably for at least 52 weeks in a human diagnosed with KL grade 2 osteoarthritis.

Thus, an object of the present invention relates to a polyacrylamide hydrogel for use in prevention and/or treatment of osteoarthritis for at least 26 weeks, preferably for at least 52 weeks in a human diagnosed with KL grade 3 osteoarthritis.

Thus, an object of the present invention relates to a polyacrylamide hydrogel for use in prevention and/or treatment of osteoarthritis for at least 52 weeks in a human diagnosed with KL grade 2 osteoarthritis.

Thus, an object of the present invention relates to a polyacrylamide hydrogel for use in prevention and/or treatment of osteoarthritis for at least 52 weeks in a human diagnosed with KL grade 3 osteoarthritis.

Thus, an object of the present invention relates to a polyacrylamide hydrogel for use in prevention and/or treatment of osteoarthritis for at least 52 weeks in a human diagnosed with KL grade 4 osteoarthritis.

Thus, an object of the present invention relates to a polyacrylamide hydrogel for use in prevention and/or treatment of osteoarthritis for at least 26 weeks, preferably for at least 104 weeks in a human diagnosed with KL grade 2 and/or KL grade 3 osteoarthritis.

Thus, an object of the present invention relates to a polyacrylamide hydrogel for use in prevention and/or treatment of osteoarthritis for at least 104 weeks in a human diagnosed with KL grade 2 osteoarthritis.

In an embodiment, the present invention relates to the polyacrylamide hydrogel for use in prevention and/or treatment of osteoarthritis for at least 104 weeks in a human diagnosed with KL grade 3 osteoarthritis.

Another object of the present invention relates to a polyacrylamide hydrogel for use in prevention and/or treatment of osteoarthritis for at least 26 weeks, preferably for at least 52 weeks, such as even more preferably for at least 104 weeks in a human diagnosed with KL grade 4 osteoarthritis.

Yet another object of the present invention relates to a polyacrylamide hydrogel for use in prevention and/or treatment of osteoarthritis for at least 104 weeks in a human diagnosed with KL grade 4 osteoarthritis.

In particular, table 11B, 12B, 13B, 16 and 17 show less WOMAC pain in KL grade 2 and or KL grade 3 subjects compared to the KL grade 4 subgroup at 26 weeks, 52 weeks and at 104 weeks.

Further, an object of the present invention relates to a polyacrylamide hydrogel for use in prevention and/or treatment of osteoarthritis for at least 26 weeks, preferably for at least 52 weeks or more preferably for at least 104 weeks in a human diagnosed with KL grade 2 and/or KL grade 3 osteoarthritis, wherein said human is female. Table 18B discloses WOMAC pain data for these particular female subgroups.

Further, an object of the present invention relates to a polyacrylamide hydrogel for use in prevention and/or treatment of osteoarthritis for at least 26 weeks, preferably for at least 52 weeks or more preferably for at least 104 weeks in a human diagnosed with KL grade 2 and/or KL grade 3 osteoarthritis, wherein said human is a male. Table 18C discloses WOMAC pain data for these particular male subgroups.

In particular, it is an object of the present invention to provide an improved or alternative way to prevent and/or treat osteoarthritis patients diagnosed with KL grade 2 and/or KL grade 3 osteoarthritis that solves or reduces the above mentioned problems of the prior art with e.g. lack of long-term or sustained pain reduction for osteoarthritis KL grade 2 and/or 3 patients. In particular, tables 11B, 12B, 13B, 16 and 17 demonstrate these surprising results.

Additionally, it is a further object of the present invention to provide an improved or alternative way to prevent and/or treat osteoarthritis patients belonging to particular sub-groups, such as patients being no more than 70 years old and/or having a BMI of less than 25 and/or in particular in male patients.

Additionally, it is a further object of the present invention to provide an improved or alternative way to prevent and/or treat osteoarthritis patients belonging to particular sub-groups, such as patients being no more than 70 years old and/or having a BMI of less than 25 and/or in particular in female patients.

Additionally, it is yet a further object of the present invention to provide an improved or alternative way to prevent and/or treat osteoarthritis patients belonging to particular sub-groups, such as patients being less than 70 years old and having a BMI categorized as overweight. In particular, table 35 demonstrate these surprising effects.

It is also an object of the present invention to provide an improved or alternative way to prevent and/or treat osteoarthritis patients belonging to particular sub-groups, such as patients being more than 70 years old and having a BMI categorized as overweight. In particular, table 36 demonstrate these surprising effects.

Yet it is a further object of the present invention to provide an improved or alternative way to prevent and/or treat osteoarthritis patients belonging to particular sub-groups, such as male patients being more than 70 years old. In particular, table 37 demonstrate these surprising effects.

Additionally, it is an object of the present invention to provide an improved or alternative way to prevent and/or treat osteoarthritis patients belonging to particular sub-groups, such as male patients having a BMI categorized as overweight. In particular, table 38 demonstrate these surprising effects.

In an embodiment, the present invention relates to the polyacrylamide hydrogel for use in prevention and/or treatment of osteoarthritis for at least 52 weeks in a human diagnosed with KL grade 2 and/or KL grade 3 osteoarthritis.

In an embodiment, the present invention relates to the polyacrylamide hydrogel for use according to the invention, wherein an at least 50% reduction in WOMAC pain subscale is obtained in said human.

An embodiment of the invention relates to the polyacrylamide hydrogel for use according to the invention, wherein about 40% of said humans obtained an at least 50% reduction in WOMAC pain subscale in said human at week 26, preferably at week 52, such as even more preferably at week 104. Preferably, about 41%, such as 42%, such as 43%, such as 44%, such as 45%, such as 50%, such as 55% of said humans obtained an at least 50% reduction in WOMAC pain subscale in said human at week 26, preferably at week 52, such as even more preferably at week 104.

In another embodiment, the present invention relates to the polyacrylamide hydrogel for use according to the invention, wherein an at least 12 points reduction in WOMAC pain subscale is obtained in said human.

An embodiment of the invention relates to the polyacrylamide hydrogel for use according to the invention, wherein about 50% of said humans obtained an at least 12 points reduction in WOMAC pain subscale in said human at week 26, preferably at week 52, such as even more preferably at week 104. Preferably, about 51%, such as 52%, such as 53%, such as 54%, such as 55%, such as 56%, such as 57%, such as 58%, such as 59%, such as 60% of said humans obtained an at least 12 points reduction in WOMAC pain subscale in said human at week 26, preferably at week 52, such as even more preferably at week 104.

In another embodiment, the present invention relates to the polyacrylamide hydrogel for use according to the invention, wherein an at least 20 points reduction in WOMAC pain subscale is obtained in said human.

An embodiment of the invention relates to the polyacrylamide hydrogel for use according to the invention, wherein about 43% of said humans obtained an at least 20 points reduction in WOMAC pain subscale in said human at week 26, preferably at week 52, such as even more preferably at week 104. Preferably, about 37%, such as 38%, such as 39%, such as 40%, such as 41%, such as 42%, such as 43%, such as 44%, such as 45%, such as 46%, such as 47%, such as 48%, such as 49%, such as 50% of said humans obtained an at least 20 points reduction in WOMAC pain subscale in said human at week 26, preferably at week 52, such as even more preferably at week 104.

In another embodiment, the present invention relates to the polyacrylamide hydrogel for use according to the invention, wherein a reduction in mean WOMAC physical function subscale is obtained in said human.

In another embodiment, the present invention relates to the polyacrylamide hydrogel for use according to the invention, wherein a reduction in mean WOMAC stiffness subscale is obtained in said human.

An embodiment of the invention relates to the polyacrylamide hydrogel for use according to the invention, wherein an about 20 points reduction in mean WOMAC physical function subscale is obtained in said human at week 26, preferably at week 52, such as even more preferably at week 104.

An embodiment of the invention relates to the polyacrylamide hydrogel for use according to the invention, wherein an about 18 points reduction in mean WOMAC stiffness subscale is obtained in said human at week 26, preferably at week 52, such as even more preferably at week 104.

In another embodiment, the present invention relates to the polyacrylamide hydrogel for use according to the invention, wherein said human is no more than 70 years old.

In another embodiment, the present invention relates to the polyacrylamide hydrogel for use according to the invention, wherein said human is less than 70 years old.

In another embodiment, the present invention relates to the polyacrylamide hydrogel for use according to the invention, wherein said human is more than 70 years old.

In another embodiment, the present invention relates to the polyacrylamide hydrogel for use according to the invention, wherein said human has a BMI of less than 25.

In another embodiment, the present invention relates to the polyacrylamide hydrogel for use according to the invention, wherein the elasticity modulus of the polyacrylamide hydrogel is more than about 10 Pa.

In another embodiment, the present invention relates to the polyacrylamide hydrogel for use according to the invention, wherein the pH of the polyacrylamide hydrogel is more than about 5.

In another embodiment, the present invention relates to the polyacrylamide hydrogel for use according to the invention, wherein the elasticity modulus of the polyacrylamide hydrogel is more than about 10 Pa and the pH of the polyacrylamide hydrogel is more than about 5.

In another embodiment, the present invention relates to the polyacrylamide hydrogel for use according to the invention, wherein the polyacrylamide hydrogel is administered by injection under sterile conditions.

In another embodiment, the present invention relates to the polyacrylamide hydrogel for use according to the invention, wherein 0.1-20 ml polyacrylamide hydrogel is administered by injection into the intraarticular cavity.

In another embodiment, the present invention relates to the polyacrylamide hydrogel for use according to the invention, wherein the polyacrylamide hydrogel is administered by injection under sterile conditions into the intraarticular cavity at least once.

In another embodiment, the present invention relates to the polyacrylamide hydrogel for use according to the invention, wherein about 6.0 ml of the polyacrylamide hydrogel is administered by injection under sterile conditions into the intraarticular cavity at least once.

In another embodiment, the present invention relates to the polyacrylamide hydrogel for use according to the invention, wherein about 3.0 ml of the polyacrylamide hydrogel is administered by injection under sterile conditions into the intraarticular cavity at least once or twice.

An embodiment of the present invention relates to the polyacrylamide hydrogel for use in prevention and/or treatment of osteoarthritis for at least 26 weeks in a human diagnosed with KL grade 2 and/or KL grade 3 osteoarthritis, wherein said human has a BMI that is categorized normal.

An embodiment of the present invention relates to the polyacrylamide hydrogel for use in prevention and/or treatment of osteoarthritis for at least 52 weeks in a human diagnosed with KL grade 2 and/or KL grade 3 osteoarthritis, wherein said human has a BMI that is categorized normal.

An embodiment of the present invention relates to the polyacrylamide hydrogel for use in prevention and/or treatment of osteoarthritis for at least 104 weeks in a human diagnosed with KL grade 2 and/or KL grade 3 osteoarthritis, wherein said human has a BMI that is categorized normal.

An embodiment of the present invention relates to the polyacrylamide hydrogel for use in prevention and/or treatment of osteoarthritis for at least 26 weeks in a human diagnosed with KL grade 2 and/or KL grade 3 osteoarthritis, wherein said human has a BMI that is categorized overweight.

An embodiment of the present invention relates to the polyacrylamide hydrogel for use in prevention and/or treatment of osteoarthritis for at least 52 weeks in a human diagnosed with KL grade 2 and/or KL grade 3 osteoarthritis, wherein said human has a BMI that is categorized overweight.

An embodiment of the present invention relates to the polyacrylamide hydrogel for use in prevention and/or treatment of osteoarthritis for at least 104 weeks in a human diagnosed with KL grade 2 and/or KL grade 3 osteoarthritis, wherein said human has a BMI that is categorized overweight.

An embodiment of the present invention relates to the polyacrylamide hydrogel for use in prevention and/or treatment of osteoarthritis for at least 26 weeks in a human diagnosed with KL grade 2 and/or KL grade 3 osteoarthritis, wherein said human has a BMI that is categorized obese.

An embodiment of the present invention relates to the polyacrylamide hydrogel for use in prevention and/or treatment of osteoarthritis for at least 52 weeks in a human diagnosed with KL grade 2 and/or KL grade 3 osteoarthritis, wherein said human has a BMI that is categorized obese.

An embodiment of the present invention relates to the polyacrylamide hydrogel for use in prevention and/or treatment of osteoarthritis for at least 104 weeks in a human diagnosed with KL grade 2 and/or KL grade 3 osteoarthritis, wherein said human has a BMI that is categorized obese.

In a further embodiment, the present invention relates to the polyacrylamide hydrogel for use in prevention and/or treatment of osteoarthritis for at least 26 weeks in a human diagnosed with KL grade 2 and/or KL grade 3 osteoarthritis, wherein said human is no more than 70 years old or preferably less than 70 years old.

In a further embodiment, the present invention relates to the polyacrylamide hydrogel for use in prevention and/or treatment of osteoarthritis for at least 52 weeks in a human diagnosed with KL grade 2 and/or KL grade 3 osteoarthritis, wherein said human is no more than 70 years old or preferably less than 70 years old.

In a further embodiment, the present invention relates to the polyacrylamide hydrogel for use in prevention and/or treatment of osteoarthritis for at least 104 weeks in a human diagnosed with KL grade 2 and/or KL grade 3 osteoarthritis, wherein said human is no more than 70 years old or preferably less than 70 years old.

In a further embodiment, the present invention relates to the polyacrylamide hydrogel for use in prevention and/or treatment of osteoarthritis for at least 26 weeks in a human diagnosed with KL grade 2 and/or KL grade 3 osteoarthritis, wherein said human is more than 70 years old.

In a further embodiment, the present invention relates to the polyacrylamide hydrogel for use in prevention and/or treatment of osteoarthritis for at least 52 weeks in a human diagnosed with KL grade 2 and/or KL grade 3 osteoarthritis, wherein said human is more than 70 years old.

In a further embodiment, the present invention relates to the polyacrylamide hydrogel for use in prevention and/or treatment of osteoarthritis for at least 104 weeks in a human diagnosed with KL grade 2 and/or KL grade 3 osteoarthritis, wherein said human is more than 70 years old.

In yet a further embodiment, the present invention relates to the polyacrylamide hydrogel for use in prevention and/or treatment of osteoarthritis for at least 26 weeks in a human diagnosed with KL grade 2 and/or KL grade 3 osteoarthritis, wherein said human has a BMI that is normal, and wherein said human is no more than 70 years old or preferably less than 70 years old.

In yet a further embodiment, the present invention relates to the polyacrylamide hydrogel for use in prevention and/or treatment of osteoarthritis for at least 52 weeks in a human diagnosed with KL grade 2 and/or KL grade 3 osteoarthritis, wherein said human has a BMI that is normal, and wherein said human is no more than 70 years old or preferably less than 70 years old.

In yet a further embodiment, the present invention relates to the polyacrylamide hydrogel for use in prevention and/or treatment of osteoarthritis for at least 104 weeks in a human diagnosed with KL grade 2 and/or KL grade 3 osteoarthritis, wherein said human has a BMI that is normal, and wherein said human is no more than 70 years old or preferably less than 70 years old.

In still a further embodiment, the present invention relates to the polyacrylamide hydrogel for use in prevention and/or treatment of osteoarthritis for at least 26 weeks in a human diagnosed with KL grade 2 and/or KL grade 3 osteoarthritis, wherein said human is a male.

In still a further embodiment, the present invention relates to the polyacrylamide hydrogel for use in prevention and/or treatment of osteoarthritis for at least 52 weeks in a human diagnosed with KL grade 2 and/or KL grade 3 osteoarthritis, wherein said human is a male.

In still a further embodiment, the present invention relates to the polyacrylamide hydrogel for use in prevention and/or treatment of osteoarthritis for at least 104 weeks in a human diagnosed with KL grade 2 and/or KL grade 3 osteoarthritis, wherein said human is a male.

In still a further embodiment, the present invention relates to the polyacrylamide hydrogel for use in prevention and/or treatment of osteoarthritis for at least 26 weeks in a human diagnosed with KL grade 2 and/or KL grade 3 osteoarthritis, wherein said human is a female.

In still a further embodiment, the present invention relates to the polyacrylamide hydrogel for use in prevention and/or treatment of osteoarthritis for at least 52 weeks in a human diagnosed with KL grade 2 and/or KL grade 3 osteoarthritis, wherein said human is a female.

In still a further embodiment, the present invention relates to the polyacrylamide hydrogel for use in prevention and/or treatment of osteoarthritis for at least 104 weeks in a human diagnosed with KL grade 2 and/or KL grade 3 osteoarthritis, wherein said human is a female.

In still a further embodiment, the present invention relates to the polyacrylamide hydrogel for use in prevention and/or treatment of osteoarthritis for at least 26 weeks in a human diagnosed with KL grade 2 and/or KL grade 3 osteoarthritis, wherein said human has a BMI that is normal, wherein said human is no more than 70 years old and wherein said human is a male.

In still a further embodiment, the present invention relates to the polyacrylamide hydrogel for use in prevention and/or treatment of osteoarthritis for at least 52 weeks in a human diagnosed with KL grade 2 and/or KL grade 3 osteoarthritis, wherein said human has a BMI that is normal, wherein said human is no more than 70 years old and wherein said human is a male.

In still a further embodiment, the present invention relates to the polyacrylamide hydrogel for use in prevention and/or treatment of osteoarthritis for at least 26 weeks in a human diagnosed with KL grade 2 and/or KL grade 3 osteoarthritis, wherein said human has a BMI that is normal, wherein said human is no more than 70 years old and wherein said human is a female.

In still a further embodiment, the present invention relates to the polyacrylamide hydrogel for use in prevention and/or treatment of osteoarthritis for at least 52 weeks in a human diagnosed with KL grade 2 and/or KL grade 3 osteoarthritis, wherein said human has a BMI that is normal, wherein said human is no more than 70 years old and wherein said human is a female.

In another aspect, the present invention relates to the polyacrylamide hydrogel for use in prevention and/or treatment of osteoarthritis for at least 26 weeks in a human, wherein said human has a BMI that is normal.

In another aspect, the present invention relates to the polyacrylamide hydrogel for use in prevention and/or treatment of osteoarthritis for at least 52 weeks in a human, wherein said human has a BMI that is normal.

In another aspect, the present invention relates to the polyacrylamide hydrogel for use in prevention and/or treatment of osteoarthritis for at least 104 weeks in a human, wherein said human has a BMI that is normal.

In yet another aspect, the present invention relates to the polyacrylamide hydrogel for use in prevention and/or treatment of osteoarthritis for at least 26 weeks in a human, wherein said human is no more than 70 years old or preferably less than 70 years old.

In yet another aspect, the present invention relates to the polyacrylamide hydrogel for use in prevention and/or treatment of osteoarthritis for at least 52 weeks in a human, wherein said human is no more than 70 years old or preferably less than 70 years old.

In yet another aspect, the present invention relates to the polyacrylamide hydrogel for use in prevention and/or treatment of osteoarthritis for at least 104 weeks in a human, wherein said human is no more than 70 years old or preferably less than 70 years old.

In another aspect, the present invention relates to the polyacrylamide hydrogel for use in prevention and/or treatment of osteoarthritis for at least 26 weeks in a human, wherein said human has a BMI that is normal, and wherein said human is no more than 70 years old or preferably less than 70 years old.

In another aspect, the present invention relates to the polyacrylamide hydrogel for use in prevention and/or treatment of osteoarthritis for at least 52 weeks in a human, wherein said human has a BMI that is normal, and wherein said human is no more than 70 years old or preferably less than 70 years old.

In another aspect, the present invention relates to the polyacrylamide hydrogel for use in prevention and/or treatment of osteoarthritis for at least 104 weeks in a human, wherein said human has a BMI that is normal, and wherein said human is no more than 70 years old or preferably less than 70 years old.

In still another aspect, the present invention relates to the polyacrylamide hydrogel for use in prevention and/or treatment of osteoarthritis for at least 26 weeks in a human, wherein said human is a male.

In still another aspect, the present invention relates to the polyacrylamide hydrogel for use in prevention and/or treatment of osteoarthritis for at least 52 weeks in a human, wherein said human is a male.

In still another aspect, the present invention relates to the polyacrylamide hydrogel for use in prevention and/or treatment of osteoarthritis for at least 104 weeks in a human, wherein said human is a male.

In still a further aspect, the present invention relates to the polyacrylamide hydrogel for use in prevention and/or treatment of osteoarthritis for at least 26 weeks in a human, wherein said human is a female.

In still a further aspect, the present invention relates to the polyacrylamide hydrogel for use in prevention and/or treatment of osteoarthritis for at least 52 weeks in a human, wherein said human is a female.

In still another aspect, the present invention relates to the polyacrylamide hydrogel for use in prevention and/or treatment of osteoarthritis for at least 104 weeks in a human, wherein said human is a female.

In another aspect, the present invention relates to the polyacrylamide hydrogel for use in prevention and/or treatment of osteoarthritis for at least 26 weeks in a human, wherein said human has a BMI that is normal, and wherein said human is no more than 70 years old or preferably less than 70 years old, and wherein said human is a male.

In another aspect, the present invention relates to the polyacrylamide hydrogel for use in prevention and/or treatment of osteoarthritis for at least 52 weeks in a human, wherein said human has a BMI that is normal, and wherein said human is no more than 70 years old or preferably less than 70 years old, and wherein said human is a male.

In another aspect, the present invention relates to the polyacrylamide hydrogel for use in prevention and/or treatment of osteoarthritis for at least 104 weeks in a human, wherein said human has a BMI that is normal, and wherein said human is no more than 70 years old or preferably less than 70 years old, and wherein said human is a male.

In a further another aspect, the present invention relates to the polyacrylamide hydrogel for use in prevention and/or treatment of osteoarthritis for at least 26 weeks in a human, wherein said human has a BMI that is normal, and wherein said human is no more than 70 years old or preferably less than 70 years old, and wherein said human is a female.

In a further another aspect, the present invention relates to the polyacrylamide hydrogel for use in prevention and/or treatment of osteoarthritis for at least 52 weeks in a human, wherein said human has a BMI that is normal, and wherein said human is no more than 70 years old or preferably less than 70 years old, and wherein said human is a female.

In another aspect, the present invention relates to the polyacrylamide hydrogel for use in prevention and/or treatment of osteoarthritis for at least 104 weeks in a human, wherein said human has a BMI that is normal, and wherein said human is no more than 70 years old or preferably less than 70 years old, and wherein said human is a female.

In a further another aspect, the present invention relates to the polyacrylamide hydrogel for use in prevention and/or treatment of osteoarthritis for at least 26 weeks, preferably such as 52, or most preferably 104 weeks in a human, wherein said human is less than 70 years old and has a BMI that is categorized as overweight.

In yet a further another aspect, the present invention relates to the polyacrylamide hydrogel for use in prevention and/or treatment of osteoarthritis for at least 26 weeks, preferably such as 52, or most preferably 104 weeks in a human, wherein said human is more than 70 years old and has a BMI that is categorized as overweight.

In yet a further another aspect, the present invention relates to the polyacrylamide hydrogel for use in prevention and/or treatment of osteoarthritis for at least 26 weeks, preferably such as 52, or most preferably 104 weeks in a human, wherein said human has a BMI that is categorized as overweight and said human is a male.

In yet a further another aspect, the present invention relates to the polyacrylamide hydrogel for use in prevention and/or treatment of osteoarthritis for at least 26 weeks, preferably such as 52, or most preferably 104 weeks in a human, wherein said human is more than 70 years old and said human is a male.

In another embodiment, the present invention relates to a method of treating osteoarthritis for at least 26 weeks, preferably for at least 52 weeks, even more preferably for at least 104 weeks in a human diagnosed with KL grade 2 and/or KL grade 3 osteoarthritis, said method comprising administering to said human a composition according to the invention.

In still another embodiment, the present invention relates to use of a composition of the invention for the preparation of a medicament for prevention and/or treatment of osteoarthritis for at least 26 weeks, preferably such as 52, or most preferably 104 weeks in a human diagnosed with KL grade 2 and/or KL grade 3 osteoarthritis.

The present invention will now be described in more detail in the following.

### DETAILED DESCRIPTION OF THE INVENTION

The hydrogel may be prepared as described in WO 02/16453, hereby incorporated by reference. In the following, the polyacrylamide hydrogel may be abbreviated PAAG.

The polyacrylamide hydrogel (PAAG) of the present invention is prepared as described in WO 02/16453, and further in WO 2012/123385, which are hereby incorporated by reference. The PAAG may comprise any embodiment of the hydrogel as described in WO 02/16453 and WO 2012/123385. The manufacture of the PAAG is further described in the Examples section. The commercial version of PAAG according to the invention is Arthrosamid® by Contura.

Preferably, the hydrogel comprises 0.5 to 25% by weight polyacrylamide, based on the total weight of the hydrogel. The hydrogel typically further comprises at least 75% by weight pyrogen-free water or saline solution, preferably pyrogen-free water.

The hydrogel is obtainable by combining acrylamide and cross-linking monomers, initiating polymerisation by radical initiation; and washing with pyrogen-free water or saline solution, the combining being in amounts and the washing being such as to give about 0.5 to 25% by weight polyacrylamide, based on the total weight of the hydrogel. The hydrogel thus obtained is both biostable and biocompatible, and is not resorbed by the body.

Typically, the hydrogel is obtained by combining acrylamide and cross-linking agent, such as N,N′-methylene bis-acrylamide, in a molar ratio of 150:1 to 1000:1. The cross-linking agent, such as N,N′-methylene bis-acrylamide, serves to provide cross-linking between polymer chains and the molar ratio may be varied to provide various cross-linking densities of the hydrogel. The conditions for obtaining the hydrogel may be modified according to, for instance, the nature of e.g. the joint, tendon, ligament, tissue into which the hydrogel is intended to be injected. The desired rheological properties, such as elasticity, may be controlled at least in part by the solid weight content of the hydrogel. The hydrogel of the invention comprises about 0.5 to 25% by weight polyacrylamide, based on the total weight of the hydrogel. In suitable embodiments of the invention, the hydrogel comprises less than 15% by weight polyacrylamide, based on the total weight of the hydrogel, preferably less than 10% by weight, more preferably less than 7.5% by weight, even more preferably less than 5%, most preferably less than 3.5% by weight polyacrylamide, based on the total weight of the hydrogel. In a preferred embodiment, the hydrogel of the invention has a solid weight contents of about 0.5 to 20% by weight polyacrylamide, based on the total weight of the hydrogel, such as about 0.5 to 15% by weight, such as about 0.5 to 10% by weight, such as about 0.5 to 5% by weight, such as about 1.0 to 5% by weight, such as about 1.5 to 5% by weight, such as about 2.0 to 5% by weight, such as about 2.5 to 5% by weight, such as about 3.0 to 5% by weight, such as about 3.5 to 5% by weight, such as about 4.0 to 5% by weight, such as about 1.0 to 4.5% by weight, such as about 1.5 to 4.5% by weight, such as about 1.5 to 4% by weight, such as about 1.5 to 3.5%, such as about 2.0 to 3.5% by weight, such as about 2.0 to 3.0% by weight, such as about 2.2 to 2.8% by weight. In a more preferred embodiment, the hydrogel of the invention has a solid weight contents of about 2.0 to 3.0% by weight of polyacrylamide, based on the total weight of the hydrogel. In an even more preferred embodiment, the hydrogel of the invention has a solid weight content of about 2.2 to 2.8% by weight polyacrylamide, based on the total weight of the hydrogel. In the present context, the term “solid weight content” is used interchangeably with the term “dry matter content”.

The combining involves the combining of the component reagents acrylamide and cross-linking agent, such as N,N′-methylene bis-acrylamide, typically degassed and typically in a manner to minimise operator contact. The reagent components may optionally be combined previously to form an inert mixture. An inert mixture is one wherein no chemical reaction proceeds among the component reagents. The combining involves combining acrylamide, cross-linking agent, such as N,N′-methylene-bis-acrylamide, and a radical initiator component to initiate polymerisation. In a suitable embodiment, an inert premixture of acrylamide, cross-linking agent, such as N,N′-methylene-bis-acrylamide, and N,N,N′,N′-tetramethylene-ethylene-diamine (TEMED) is combined with an ammonium persulfate (AMPS) initiator solution. However, the components may be combined as singularities or as alternative plural premixtures.

Acrylamide and cross-linking agent, such as N,N′-methylene-bis-acrylamide, are suitably combined in a molar ratio of about 150:1 to 1000:1, typically about 150:1 to 900:1, preferably about 175:1 to 800:1, more preferably about 200:1 to 600:1, most preferably from 250:1 to 600:1. As shown in Tables 2 and 3, hydrogels of differing solid-weight content and rheological properties may be controllably prepared. The hydrogel having the desired rheological characteristics has been obtained by combining acrylamide and N,N′-methylene-bis-acrylamide in a ratio of about 250:1, about 260:1, about 270:1, about 280:1, about 290:1, about about 300:1, about 310:1, about 320:1, about 330:1, about 340:1, about 350:1, about 360:1, about 370:1, about 380:1, about 390:1, about 400:1, about 410:1, about 420:1, about 430:1, about 440:1, about 450:1, about 460:1, about 470:1, about 480:1, about 490:1 and about 500:1.

Particularly in the embodiment wherein the hydrogel is injected into a joint, tendon or ligament the elasticity of the hydrogel is of great relevance. Persons skilled in the art will be aware of how to obtain a hydrogel with a suitable elasticity for the intended use. See also the examples below, which describe preparation of hydrogels with low, medium and high elasticity.

It is important that the PAAG is stable, physically and chemically after formation. In fact, degradation products from the polymerization process (sulphate) is below 4 ppm and once the polymerization process is to an end, the PAGG remains physically and chemically stable, also after having been injected into tissue. In tissue, the gel also remains chemically and physically stable, and does not release leachable products, as it does not degrade in tissue at all—it is permanent. The PAAG hydrogel does not degrade over time (i.e. it is permanent) and it allows fibre and vessel ingrowth. Thus, the PAAG hydrogel of the present invention is non-degradable, safe to use and non-toxic i.e. it does not release toxic components over time.

The hydrogel comprises at least 75% by weight pyrogen-free water or saline solution, preferably pyrogen-free water. In a suitable embodiment of the invention, the hydrogel comprises at least 80% by weight pyrogen-free water or saline solution, preferably at least 85%, more preferably at least 90%, even more preferably at least 95% by weight pyrogen-free water or saline solution.

A suitable saline solution has an osmolarity similar to that of interstitial fluid. Suitable saline solutions include but are not limited to the group comprising 0.25-1% aqueous sodium chloride, a Ringer-Lockart solution, an Earle solution, a Hanks solution, an Eagle medium, a 0.25-1% glucose solution, a potassium chloride solution, and a calcium chloride solution. In a preferred embodiment, the saline solution is a 0.8-1% aqueous sodium chloride solution, such as a 0.8, 0.9 or 1% aqueous sodium chloride solution, most preferably about 0.9% aqueous sodium chloride.

As will be obvious to the person skilled in the art, in the embodiment wherein saline solution is used either for the preparation of the PAAG and/or for the washing of the PAAG, the solid-weight content of the PAAG will be higher than the contribution made by the polyacrylamide, but typically not more than an additional 1%.

In a particularly suitable embodiment of the invention, the hydrogel comprises about 2.5% by weight polyacrylamide, based on the total weight of the hydrogel and about 97.5% pyrogen-free water.

Pyrogen-free water or saline solution is used for washing the hydrogel in a washing process. The washing process serves, in part, to remove all but trace amounts of the monomers acrylamide and cross-linking agent, such as N,N′-methylene-bis-acrylamide. These monomers are toxic to the patient as well as detrimental to the stability of the hydrogel. The washing process is preferably such that the concentrations of the remaining monomers acrylamide and cross-linking agent, such as N,N′-methylene-bis-acrylamide, are below 50 ppm, more preferably below 40 ppm, such as below 30 ppm, most preferably below 20 ppm, typically below 10 ppm, particularly preferably below 5 ppm or even more preferably below 1.5 ppm.

The hydrogel according to the present invention may contain a cross-linking agent selected from the group consisting of N,N′-methylene-bis-acrylamide, N,N′-ethylene-bis-acrylamide, ethylene-bis (oxyethylene nitril)-tetracetic oxide, ethylene-bis-(oxyethylene nitril) tetracetic acid, and mixtures thereof. In one embodiment, said cross-linking agent is selected from the group consisting of N,N′-methylene-bis-acrylamide, N,N′-ethylene-bis-acrylamide, and mixtures thereof. In a further embodiment, said cross-linking agent is N,N′-methylene-bis-acrylamide.

As noted above, one aspect of the present invention relates to a polyacrylamide hydrogel for use in prevention and/or treatment of osteoarthritis for at least 26 weeks, such as preferably for at least 52 weeks in a human diagnosed with KL grade 2 and/or KL grade 3 osteoarthritis.

As discussed above, osteoarthritis (OA) is a painful, debilitating joint disease with no known cure. It is characterized by heat, pain, swelling, crepitus (a crackling, crinkly, or grating feeling or sound under the skin), and a decreased range of motion in affected joints. In humans it affects the hands, knees, hips, spine and other joints. The stage of the OA in patients can be divided into subgroups by physicians, i.e. KL grade 0-4.

KL is short for the “Kellgren-Lawrence Classification of Osteoarthritis”. KL is a well-known grading scale used by physicians to diagnose the severity of OA in patients by radiological assessment. This grading scale allows physicians to divide patients into well-defined sub-groups. Accordingly, the OA severity of a patient is evaluated and patients are divided into separate and individual KL grade groups according to the following classification scale:
KL grade 0 (normal): no radiographic feature of osteoarthritis.
KL grade 1 (doubtful/questionable): doubtful joint space narrowing and possible osteophytic lipping.
KL grade 2 (minimal): definite osteophytes and possible joint space narrowing.
KL grade 3 (moderate): moderate multiple osteophytes, definite narrowing of joint space and some sclerosis and possible deformity of bone ends.
KL grade 4 (severe): large osteophytes, marked joint space narrowing, severe sclerosis and definite body deformity.

Hence, according to the invention, e.g. “KL grade 2 and/or 3” refers to the Kellgren-Lawrence Classification of Osteoarthritis grade 2 and/or 3 and thus refers to the sub-group of patients suffering from minimal or moderate OA. For short, sometimes KL gade 2 is simply mentioned as “KL 2”, and KL grade 3 as “KL 3” and so forth.

Referring to “KL grade 2-3” or “KL 2-3” means the added group of the individual subgroups KL grade 2 and KL grade 3 pooled into one.

Surprisingly, the present inventors have found that polyacrylamide hydrogel (PAAG) is particularly useful in the prevention and/or treatment of OA in individual subgroups of humans suffering from OA, such as subgroups of humans diagnosed with KL grade 2 and/or KL grade 3 osteoarthritis, as is clearly demonstrated in the data, e.g. tables 11-13, 16-17.

Accordingly, in a preferred embodiment, the present invention relates to the polyacrylamide hydrogel for use in prevention and/or treatment of osteoarthritis for at least 52 weeks in a human diagnosed with KL grade 2 and/or KL grade 3 osteoarthritis. The data clearly show such effect for at least 52 weeks, e.g. in tables 11-13, 16-17.

Accordingly, in a preferred embodiment, the present invention relates to the polyacrylamide hydrogel for use in prevention and/or treatment of osteoarthritis for at least 104 weeks in a human diagnosed with KL grade 2 and/or KL grade 3 osteoarthritis.

Surprisingly, the present inventors have found that polyacrylamide hydrogel (PAAG) is particularly useful in the prevention and/or treatment of OA in the individual subgroup of humans diagnosed with KL grade 2 OA, as is clearly demonstrated in the data, e.g. tables 11-13, 16-17.

Surprisingly, the present inventors have found that polyacrylamide hydrogel (PAAG) is particularly useful in the prevention and/or treatment of OA in the individual subgroup of humans diagnosed with KL grade 3 OA, as is clearly demonstrated in the data, e.g. tables 11-13, 16-17.

In an embodiment, the present invention relates to the polyacrylamide hydrogel for use in prevention and/or treatment of osteoarthritis for at least 4 weeks in a human diagnosed with KL grade 2 and/or KL grade 3 osteoarthritis, or such as at least 12 weeks, such as at least 78 weeks, such as about 1.5 years, such as at least 104 weeks or about 2 years, such as up to at least about 260 weeks or at least about 5 years in a human diagnosed with KL grade 2 and/or KL grade 3 osteoarthritis.

According to the invention, pain is assessed by use of The Western Ontario and McMaster Universities Arthritis Index (WOMAC). It is a proprietary set of standardized questionnaires used by health professionals to evaluate the condition of patients with osteoarthritis of the knee and hip, including pain, stiffness, and physical functioning of the joints. Hence, the WOMAC is used to assess pain in the knee, hip, elbow, the metacarpal-phalangeal and interphalangeal joints in hands and feet, the sesamoid joint and/or the temporomandibular joint in humans.

In an embodiment, the present invention relates to the polyacrylamide hydrogel for use according to the invention, wherein an at least 50% reduction in WOMAC pain subscale is obtained in said human, or such as an at least 45% reduction in WOMAC pain subscale, or an at least 40% reduction in WOMAC pain subscale, or an at least 35% reduction in WOMAC pain subscale, or an at least 30% reduction in WOMAC pain subscale, or s an at least 25% reduction in WOMAC pain subscale, or an at least 20% reduction in WOMAC pain subscale, or an at least 15% reduction in WOMAC pain subscale, or an at least 10% reduction in WOMAC pain subscale, or an 10-50% reduction in WOMAC pain subscale, or an 20-50% reduction in WOMAC pain subscale, or an 30-50% reduction in WOMAC pain subscale or an 30-40% reduction in WOMAC pain subscale. It may be noted that an at least 30-40% reduction in WOMAC pain subscale is considered a very satisfying result.

Thus, an embodiment of the invention relates to the polyacrylamide hydrogel for use in prevention and/or treatment of osteoarthritis for at least 26 weeks, preferably such as at least 52 weeks, such as even more preferably at least 104 weeks in a human diagnosed with KL grade 2 and/or KL grade 3 osteoarthritis, wherein an at least 50% reduction in WOMAC pain subscale is obtained in said human. Data to demonstrate the at least 50% reduction effects are shown e.g. in tables 11A and 11B.

WOMAC pain subscale: The WOMAC consists of three subscales: pain (five questions), stiffness (two questions), and physical function (17 questions). The subscale scores can vary, with pain ranging from 0 to 20 points; stiffness, 0 to 8 points; and physical function, 0 to 68 points. Accordingly, higher scores indicate worse pain, stiffness, and functional limitations.

In another embodiment, the present invention relates to the polyacrylamide hydrogel for use according to the invention, wherein an at least 12 points reduction in WOMAC pain subscale is obtained in said human, or such as at least 5 points, such as at least 7 points, such as at least 9 points, such as at least 11 points, such as at least 13 points, such as at least 15 points, such as at least 17 points, such as at least 19 points, or such as 5-20 points, such as 7-20 points, such as 9-20 points, such as 11-20 points, such as 13-20 points, such as 15-20 points, such as 17-20 points reduction in WOMAC pain subscale is obtained in said human. Data to demonstrate the at least 12 point effects are shown e.g. in tables 11A and 11B.

In a preferred embodiment, the present invention relates to the polyacrylamide hydrogel for use according to the invention, wherein an at least 20 points reduction in WOMAC pain subscale is obtained in said human. Data to demonstrate the at least 20 point effects are shown e.g. in tables 13A and 13B.

Preferably, an at least 12 or 20 points reduction in WOMAC pain subscale is obtained in a human diagnosed with KL grade 2 and/or KL grade 3 osteoarthritis. Thus, an embodiment of the invention relates to the polyacrylamide hydrogel for use in prevention and/or treatment of osteoarthritis for at least 26 weeks or preferably at least 52 weeks in a human diagnosed with KL grade 2 and/or KL grade 3 osteoarthritis to obtain an at least 12 or 20 points reduction in WOMAC pain subscale.

Thus, an embodiment of the invention relates to the polyacrylamide hydrogel for use in prevention and/or treatment of osteoarthritis for at least 104 weeks in a human diagnosed with KL grade 2 and/or KL grade 3 osteoarthritis to obtain an at least 12 or 20 points reduction in WOMAC pain subscale.

Another embodiment of the invention relates to the polyacrylamide hydrogel for use according to the invention, wherein a reduction in mean WOMAC physical function subscale is obtained in said human. Data to demonstrate the reduction in mean WOMAC physical function subscale are shown e.g. in table 16.

Another embodiment of the invention relates to the polyacrylamide hydrogel for use according to the invention, wherein a reduction in mean WOMAC stiffness subscale is obtained in said human. Data to demonstrate the reduction in mean WOMAC stiffness subscale are shown e.g. in table 17.

In another embodiment, the present invention relates to the polyacrylamide hydrogel for use according to the invention, wherein said human is no more than or equal to preferably 70 years old, such as no more than or equal to 80 years old, such as no more than or equal to 65 years old, such as no more than or equal to 60 years old, such as no more than or equal to 50 years old, such as no more than or equal to 45 years old, such as no more than or equal to 40 years old, such as no more than or equal to 35 years old, such as 50-59 years old, such as 60-69 years old, such as 70-79 years old. In a preferred embodiment, said human is less than 70 years old, or more than 70 years old.

Thus, an embodiment of the invention relates to the polyacrylamide hydrogel for use in prevention and/or treatment of osteoarthritis for at least 26, such as preferably for at least 52 or such as even more preferably for at least 104 weeks in a human diagnosed with KL grade 2 and/or KL grade 3 osteoarthritis, wherein said human is less than 70 years old or more than 70 years old.

Data to demonstrate the effects of said human being less than 70 years old, or more than 70 years old are shown e.g. in table 32.

In another embodiment, the present invention relates to the polyacrylamide hydrogel for use according to the invention, wherein said human has a BMI of less than 25, such as less than 23, such as less than 21, such as less than 19, such as less than 27, such as less than 29, such as less than 31, preferably such as 18.5 to 24.9 (normal weight BMI), such as 25 to 29.9, or such as 30 or more. In a further embodiment, said human has a BMI that is categorized as normal weight. BMI that is categorized as normal weight is used interchangeably with BMI that is normal (BMI: 18.5 to 24.9).

Body mass index (BMI) is a value derived from the mass (weight) and height of a human. The BMI is defined as the body mass divided by the square of the body height, and is expressed in units of kg/m2, resulting from mass in kilograms and height in metres.

The BMI is a convenient rule of thumb used to broadly categorize a person as underweight, normal weight, overweight, or obese based on tissue mass (muscle, fat, and bone) and height. Major adult BMI classifications are underweight (under 18.5 kg/m2), normal weight (18.5 to 24.9), overweight (25 to 29.9), and obese (30 or more).

Surprisingly, the present inventors have found that polyacrylamide hydrogel (PAAG) is particularly useful in the prevention and/or treatment of osteoarthritis in individual subgroups of humans suffereing from OA, such as subgroups of humans diagnosed with KL grade 2 and/or KL grade 3 osteoarthritis, wherein said human has a BMI that is categorized as normal, preferably such as categorized overweight, or such as categorized obese. Data to demonstrate the BMI effects of said human are shown e.g. in table 19-20.

In another embodiment, the present invention relates to the polyacrylamide hydrogel for use according to the invention, wherein the elasticity modulus of the polyacrylamide hydrogel is more than about 10 Pa, such as more than about 20 Pa, such as more than about 30 Pa, such as more than about 40 Pa, such as more than about 50 Pa, such as more than about 60 Pa, such as more than about 70 Pa, such as more than about 80 Pa, such as more than about 90 Pa, such as more than about 100 Pa, such as 40-70 Pa, such as such as 40-150 Pa, such as such as 40-200 Pa.

In a preferred embodiment, the hydrogel of the invention has an elasticity modulus of about 10 to 200 Pa, such as about 15 to 180 Pa, such as about 20 to 150 Pa, such as 30 to 120 Pa, such as 40 to 120 Pa, such as 50 to 120 Pa or such as about 1 to 200 Pa, such as about 2 to 175 Pa, typically about 5 to 150 Pa, such as 10 to 100 Pa, such as 20 to 100 Pa, such as 30 to 100 Pa, such as 40 to 100 Pa such as 50 to 100 Pa. Persons skilled in the art will be aware of how to obtain a hydrogel with a suitable elasticity for the intended use. See also the examples below, which describe preparation of hydrogels with low, medium and high elasticity.

In the range of 35 Pa and up to at least 185 Pa the crosslinked polyacrylamide hydrogel behaves as an ideal elastic material whereas in the lower end of less than 10 Pa the hydrogel become more viscous with insufficient elasticity for ideal syringeability properties.

The elasticity modulus (G′) is a measure for the total elastic property of a material (in contrast to the flowrate of the material). The value of the elasticity modulus is a sum of the various elements contributing to the elasticity, including particles size, the degreed of cross lining, the molecular forces between the particles etc.

To measure the elasticity modulus a well-defined volume of sample is placed between two plates where one plate is stationary and the motion pattern of the other can be controlled. During measurement, the dynamic plate moves with a small-amplitude sinusoidal oscillation while plotting the shear force required for this deformation to appear. The experiment can be run at different frequencies. However, for the purposes of establishing the correct measurement the frequency is 1 Hz. When the plate is oscillated, the microstructures of the hydrogel are exposed to stress and seek to relax the stress to minimize the overall energy of the hydrogel. The slower the deformation, the more relaxation of microstructures is possible and thus the lower elasticity modulus. In other words, if a fast deformation is applied, then many microstructures of the hydrogel will not be able to relax their stresses within the given period (scaling with the inverse frequency). The hydrogel will thus behave more elastic at high frequency than at low frequency. Measurements are performed under controlled environmental conditions.

In another embodiment, the present invention relates to the polyacrylamide hydrogel for use according to the invention, wherein the pH of the polyacrylamide hydrogel is more than about 4, such as preferably more than about 5, such as more than about 6, such as more than about 7, such as more than about 8, such as more than about 9, such as 5-10, or 5.5-9.5, 6.0-9.0, 6.5-8.5, 7.0-8.5, 7.5-8.5 or pH 7.8-8.2.

In another embodiment, the present invention relates to the polyacrylamide hydrogel for use according to the invention, wherein the elasticity modulus of the polyacrylamide hydrogel is more than about 10 Pa and the pH of the polyacrylamide hydrogel is more than about 5.

In another embodiment, the present invention relates to the polyacrylamide hydrogel for use according to the invention, wherein the polyacrylamide hydrogel is administered by injection under sterile conditions.

The injection of the PAAG may be performed under local anaesthesia, but local anaesthesia is not necessarily required. However, the procedure is preferably performed under sterile conditions. Any hair covering the injection area is cropped and the skin thoroughly rinsed e.g. with chlorhexidine and ethanol (e.g. 3 times interchangeably). Then, the cannula is inserted into the joint cavity and it is checked by aspiration that it is placed properly intraarticularly. Generally, the joint is emptied for at least the amount of liquid which it has been decided to inject and the desired amount of the PAAG is then injected. An antibiotic may be included in the PAAG in order to prevent iatrogenic infection of the joint.

In another embodiment, the present invention relates to the polyacrylamide hydrogel for use according to the invention, wherein 0.1-20 ml polyacrylamide hydrogel is administered by injection into the intraarticular cavity.

An appropriate amount of polyacrylamide hydrogel will be in the range of 0.1 ml to 20 ml, such as 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18 or 19 ml. Preferably, an amount of 1-2 ml or more, such as 1-3, such as 1-4, such as 1-5, such as 1-6, such as 2-7, such as 3-6, such as 4-6, such as 4-7, such as 5-7, such as 5-6, such as 1-7, such as 1-8, such as 1-9, such as 1-10, such as 1-11, such as 1-12, such as 1-13, such as 1-14, such as 1-15, such as 1-16, such as 1-17, such as 1-18, such as 1-19 ml is administered. However, it is not advisable to inject so much hydrogel that the joint is expanded. The exact amount will be decided by the treating physician on the basis of the size of the joint and the severity of the osteoarthritis but typically about 6.0 ml is injected. Accordingly, most preferred is an amount of about 6 ml (one injection) or about 3 ml (injected once or twice). In a preferred embodiment, said about 3.0 ml or 6.0 ml second injection is performed within about 14 days following the first injection, such as within about 21 days following the first injection, such as within about 1 or about 2 month following the first injection. Preferably, the second injection is performed between about day 14 and 1 month following the first injection, or between about day 14 and 2 month following the first injection, or between about day 14 and 3 month following the first injection.

In another embodiment, the present invention relates to the polyacrylamide hydrogel for use according to the invention, wherein the polyacrylamide hydrogel is administered by injection under sterile conditions into the intraarticular cavity at least once, such as twice, such as three times, such as four times, such as 5 times, such as 6 times, such as 7 times, such as 8 times, such as 9 times, such as 10 times. The administration can be performed during a period of several years, such as during a period of 1 year, 2, 3, 4, 5, 6, 7, 8, 9 or 10 years.

In another embodiment, the present invention relates to the polyacrylamide hydrogel for use according to the invention, wherein preferably about 6.0 ml or about 3.0 ml of the polyacrylamide hydrogel is administered by injection under sterile conditions into the intraarticular cavity at least once. It may be advantageous for patients to receive only one injection of about 6 ml polyacrylamide hydrogel according to the invention to improve patient acceptability, feasibility of treatment and reduce the risk of potential side effects related to repeated injections.

In another preferred embodiment of the invention, the joint or joints of the human which is/are to be treated is the knee, hip, elbow, the metacarpal-phalangeal and interphalangeal joints in hands and feet, the sesamoid joint and/or the temperomandibular joint.

In another embodiment, the present invention relates to the polyacrylamide hydrogel for use according to the invention, wherein about 3.0 ml of the polyacrylamide hydrogel is administered by injection under sterile conditions into the intraarticular cavity at least once or twice. It may be advantageous for patients to receive only one injection of about 3.0 ml polyacrylamide hydrogel according to the invention to reduce the risk of potential side effects related to repeated injections and lower the cost by only having to pay for 3.0 ml injection.

In a preferred embodiment, said about 3.0 ml or 6.0 ml second injection is performed within about 14 days following the first injection, such as within about 21 days following the first injection, such as within about 1 or about 2 month following the first injection. Preferably, the second injection is performed between about day 14 and 1 month following the first injection, or between about day 14 and 2 month following the first injection, or between about day 14 and 3 month following the first injection.

Surprisingly, the present inventors have found that polyacrylamide hydrogel (PAAG) is particularly useful in the prevention and/or treatment of osteoarthritis in individual subgroups of humans suffereing from OA, such as subgroups of humans diagnosed with KL grade 2 and/or KL grade 3 osteoarthritis, wherein said human is female. Data to demonstrate the gender effects of said human are shown e.g. in tables 18A, 18B.

Thus, in another embodiment, the present invention relates to the polyacrylamide hydrogel for use according to the invention, wherein said human is a male. Surprisingly, the present inventors have found that polyacrylamide hydrogel (PAAG) is particularly useful in the prevention and/or treatment of osteoarthritis in individual subgroups of humans suffereing from OA, such as subgroups of humans diagnosed with KL grade 2 and/or KL grade 3 osteoarthritis, wherein said human is male. Data to demonstrate the gender effects of said human are shown e.g. in table 18A, 18C.

Thus, in another embodiment, the present invention relates to the polyacrylamide hydrogel for use according to the invention, wherein said human is a female.

In another embodiment, the present invention relates to the polyacrylamide hydrogel for use according to the invention, wherein said polyacrylamide hydrogel does not contain silver ions. Silver is the chemical element with the symbol Ag. The presence of silver ions in a polyacrylamide hydrogel decreases the elasticity modulus of the gel thereby making it less suitable for intraarticular injection according to the invention.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1—Preparation of Hydrogel (PAAG)

### Preparation of Hydrogel:

The PAAG is a polyacrylamide gel manufactured by a polymerisation of the monomers of acrylamide (AM) and N,N′-methylene-bis-acrylamide (bisAM). The finished product may have different elasticity modules.

The hydrogel typically contains approximately 95% water. The concentration of the monomers acrylamide and N,N′-methylene-bis-acrylamide has been shown to be less than 10 ppm and is adequate for the desired stability of the final product, often less than 5 ppm.

The finished product must conform with respect to pH, absence of heavy metals, refractive index, stability, absence of pyrogens, and must be sterile, practically inert, and be substantially free of monomers.

### Preparation 1.1

The synthetic preparation suitably involves the following operations:
1. Two mixtures, A1 and A2, are prepared. A1 comprises water, acrylamide, N,N′-methylene-bis-acrylamide, N,N,N′,N′-tetramethylene-ethylene-diamine (TEMED).
A2 comprises water and ammonium persulfate;
2. The two mixtures are combined in the following ratio: 1990 mL of A1 and 10 mL of A2 and kept at 45° C. and degassed with nitrogen for 20 seconds;
3. The reaction mixture is cast into several 100 ml beakers;
4. Polymerisation is allowed to occur for 0.5 to 1.5 hours;
5. The gel is demolded;
6. Residual monomers are extracted and with equilibration in WFI water for 92 hours, changing the water several times, typically 8 times during the 92 hours;
7. The purified gels are homogenised by grinding with a vertically oscillating grid;
8. A syringe is filled with the homogenised gel material;
9. Autoclavation of the syringe

A typical method for preparing the hydrogel may be summarised as:

### Preparation 1.2

### Process Summary

The gel is prepared by mixing an aqueous monomer solution of acrylamide (AM) and N,N′-methylene-bis-acrylamide (bisAM) as cross-linker with N,N, N′,N′-tetramethylene ethylene diamine (TEMED) as co-initiator and ammonium persulfate (AMPS) as free-radical initiator (redox-system). By degassing a bulk solution with nitrogen, polymerisation starts. After final polymerisation the gel is transferred into a washing tank with net trays onto which the gel is placed. During water washing, the gel swells and monomer residues are extracted. The swollen gel is fed and evacuated in a filling unit having the gel delivered in a syringe, which is autoclaved. Two alternate formulations have been prepared, a lower- and a higher-end elasticity formulation.

TABLE 1

| | lower end | higher end |
|---|---|---|
| Chemical constituent | elasticity | elasticity |
| acrylamide | 502 g | 547 g |
| N,N′-methylene-bis- | 2.2 g | 4.6 g |
| acrylamide | | |
| TEMED | 3.0 g | 2.6 g |
| AMPS | 5.4 g | 5.0 g |
| Non-pyrogenic water | Add 10 litre | Add 10 litre |

The above are typical preparations of the hydrogel and may be adjusted within certain ranges.

### Preparation 1.3

Polyacrylamide Formulations from Inline Cross-Linking Process

A particularly interesting method of preparing the hydrogels of the invention involves an inline cross-linking process. Two individual and eventually degassed flows, one being a pre-mix of acrylamide, N,N′-methylene-bis-acrylamide (the cross-linker) and TEMED, the other being the AMPS initiator solution, are pumped into a static mixer for mixing, chemical initiation and subsequent extrusion downstream into a pipe reactor made of Teflon or steel in which the polymerisation occurs. Washing of the gel is simplified due to high surface area of gel from reactor.

By selecting monomer, cross-linker and initiator concentrations and their relative molar ratios, and by regulating the two flow rates and the polymerisation temperatures, it is possible to produce gels that are varying in degree of crosslinking and in solids content.

### Preparation 1.4

The reagents were combined in ratios described in Tables 2, 3 and 4, and washed as described in the Tables (with pyrogen-free water unless indicated otherwise) to give low, medium, and high elasticity formulations. Hydrogels with solid weight contents between 0.5 and 25% polyacrylamide were prepared.

TABLE 2
Process parameters and features of resulting gel: low elasticity formulations

| | Iv1 | Iv2 | Iv3 | Iv4 | Iv5 | Iv6 | Iv7 ^{d} | Iv8 ^{e} |
|---|---|---|---|---|---|---|---|---|
| washing time | a) | 19.5 | 73.75 | 92 | 94.3 | 72.8 | 93.6 | 93.9 |
| (hrs) | | | | | | | | |
| dry matter ⁱ | 2.55 | 2.08 | 2.63 | 2.87 | 2.89 | 3.15 | 3.68 | 3.17 |
| (%) | | 2.36 | 2.58 | 2.67 | 2.82 | 2.90 | 3.57 | 3.52 |
| | | | 2.09 | | | | | |
| molar ratio | b) | 976 | 700 | 488 | 366 | 3239 | 488 | 488 |
| AM:bisAM | | | | | | | | |
| molar ratio AM + | 252 | 252 | 253 | 251 | 252 | 249 | 252 | 252 |
| bisAM:TEMED | | | | | | | | |
| molar ratio AM + | 298 | 299 | 298 | 298 | 298 | 299 | 298 | 298 |
| bisAM:AMPS | | | | | | | | |
| residual | c) | 89 | 5 | 2.97 | 2 | 5 | 1.4 | 0.97 |
| monomer in | | | | | | | | |
| ppm | | | | | | | | |
| elasticity G′ in | 0.16 | 5.23 | 14.3 | 26.6 | 57.05 | 71.7 | 39.2 | 28.5 |
| Pa | | | 20.1 | | | | | |
| gelation time | liquid | highly | 12 | 2 | 2 | 2 | 2.5 | 2.5 |
| (min) | | viscous | | | | | | |
| | | liquid | | | | | | |

| | Iv9 | Iv10 | Iv11 | Iv11 | Iv12 |
|---|---|---|---|---|---|
| washing time (hrs) | 121 | 96.4 | | | |
| dry matter | 2.18 | (5.10)^{f} | (10.2)^{f} | (10.1)^{f} | (20.2)^{f} |
| (%) | | | | | |
| molar ratio | 701 | 701 | 488 | 488 | 488 |
| AM:bisAM | | | | | |
| molar ratio AM + | 252 | 252 | 252 | 504 | 2016 |
| bisAM:TEMED | | | | | |
| molar ratio AM + | 298 | 298 | 298 | 596 | 2385 |
| bisAM:AMPS | | | | | |
| residual monomer in ppm | 0.97 | | | | |
| elasticity G′ in Pa | 28.5 | 11.1 | (911)^{g} | (1240)^{g} | (9460)^{g} |
| gelation time (min) | | 3.17 | 0.00 | 1.21 | 3.5 ^{h} |

a) material was liquid so washing was a dilution
b) infinite
c) since washing was not an extraction but a dilution, the residual monomer was merely decreased by the dilution factor (508 ppm to 254 ppm).
^{d} casting and washing done using 0.9% NaCl aqueous solution
^{e} casting with water; washing done using 0.9% NaCl aqueous solution
^{f}pre-wash values-washing typically reduces value by 30-55%
^{g}pre-wash values-washing typically reduces value by 20-40%
^{h} highly notch sensitive
ⁱ variations in values may be due to measurement performance techniques or to location in the batch from which sample was taken

TABLE 3
Process parameters and features of resulting gel:
medium elasticity formulations

| | mv1 | mv2 | mv3 | mv4 | mv5 |
|---|---|---|---|---|---|
| washing time (hrs) | 97 | 211.5 | 96 | 94.8 | 90.3 |
| dry matter (%) | 3.14 | 2.49 | 3.25 | 3.29 | 3.22 |
| molar ratio | 310 | 310 | 290 | 289 | 289 |
| AM:bisAM | | | | | |
| molar ratio | 252 | 252 | 252 | 251 | 252 |
| AM + BISAM:TEMED | | | | | |
| molar ratio | 299 | 299 | 299 | 299 | 299 |
| AM + BISAM:APS | | | | | |
| residual monomer in | 1.6 | | 1.5 | | |
| ppm | | | | | |
| elasticity G′ in Pa | 108.5 | | 129 | 133.5 | |
| gelation time | 2.5 | 2.5 | 2.18 | | |
| (min) | | | | | |

TABLE 4
Process parameters and features of resulting gel:
high elasticity formulations

| | hv1 | hv2 | hv3 | hv4 | hv5 |
|---|---|---|---|---|---|
| washing time (hrs) | 119.5 | 516 | 122 | 95.5 | 116.7 |
| dry matter (%) | 3.47 | 2.5 | 3.56 | 3.83 | 3.42 |
| molar ratio | 260 | 260 | 260 | 260 | 260 |
| AM:bisAM | | | | | |
| molar ratio | 315 | 315 | 604 | 313 | 314 |
| AM + bisAM:TEMED | | | | | |
| molar ratio | 376 | 376 | 755 | 375 | 376 |
| AM + bisAM:AMPS | | | | | |
| residual monomer in | 0.2 | | | | |
| ppm | | | | | |
| elasticity G′ in Pa | 343 | 274 | | 314.5 | |
| gelation time | 2.18 | 2.18 | 7.5 | | |
| (min) | | | | | |

### Example 2—Clinical Investigation and Results Obtained Therefrom

A multi-centre, randomised, controlled, double-blind clinical investigation of intra-articular polyacrylamide hydrogel injection in subjects with knee osteoarthritis followed by an open label extension study was performed. The study protocol was approved and registered at www.clinicaltrials.gov (ref. no. NCT04179552).

The subjects enrolled in the investigation were suffering from mild to severe knee OA. Thus, the subjects were categorised into KL grades 2-4 according to the standard Kellgren-Lawrence classification. Individual subgroups of KL grades were investigated as well as KL grade groups. Thus KL grade 2, KL grade 3, KL grade 4, KL grade 2-3, and KL grade 2-4 groups have been investigated at timepoints 26, 52 and 104 weeks.

The subjects were injected with either one injection of 6 mL intra-articular PAAG-OA or with one injection of 6 mL Synvisc-One at time zero. PAAG-OA (Arthrosamid®) is defined in Example 1, and is a commercial polyacrylamide hydrogel from Contura, while Synvisc-One (hylan G-F 20) is a commercial hyaluronic acid gel from Sanofi-Aventis.

The participating subjects were followed from baseline to week 104 answering the WOMAC questionnaire prior to the injection as well as on the following weeks: 4, 12/13, 26, 52 and 104.

The study protocol was expanded over time from 52 weeks, allowing measurements on 2 years (104 weeks). Data collection will continue up till 5 years.

### WOMAC: Western Ontario and McMaster Universities Osteoarthritis Index.

The WOMAC pain subscale is the sum of the first 5 questions from the WOMAC Index: pain during walking, pain using stairs, pain in bed, pain sitting or lying, and pain standing.

The subscale can take on values from 0 to 20 where higher scores indicate higher pain. The subscale will be transformed (normalised) to range from 0-100 in the following way

Transformed pain subscale=(pain subscale*0.5)*10

### Post Hoc Analysis:

Question 1 from WOMAC questionnaire (walking on flat surface)
Percentage change in WOMAC endpoints and PGA (Patients Global Assessment) of disease impact.

### Study Design Definitions:

Intention-to-Treat (ITT) analysis set: The ITT analysis set will consist of all randomised subjects irrespective of whether the subject received study intervention or the subject's compliance with the study protocol. The subjects will be included as randomised.

Full analysis set (FAS): The FAS will consist of all randomised subjects, who received study treatment and who have WOMAC pain subscale assessed at baseline and at least one post-baseline assessment. The subjects will be included as randomised.

Per Protocol (PP) analysis set: The PP analysis set is defined as all subjects in the FAS, meeting all inclusion criteria, and who do not have any protocol deviations of clinical or statistical significance.

Safety analysis set: The safety analysis set is defined as subjects who have received a study treatment. The subjects will be included as actually treated.

Overview of Results from Clinical Study from Example 2

### Clinical Results Overview:

Tables 5-7 show selected demographics from the study.
Tables 8-10 show pain data from studied subjects (total KL grade 2-4) at timepoint ½ and 1 years i.e.
   Table 8: at least 50% reduction in pain.
   Table 9: at least 12 point reduction in pain.
   Table 10: at least 20 point reduction in pain.
Tables 11-13 show pain data from KL subgroups KL 2, 3, 4, 2-3 and 2-4 at timepoint ½, 1 and 2 years i.e.
   Table 11: at least 50% reduction in pain.
   Table 12: at least 12 point reduction in pain.
   Table 13: at least 20 point reduction in pain.
Tables 14 and 15 show further details of the data from the clinical study.
Tables 16-17 show pain data from the pain subscales “physical function” and “stiffness”
Tables 18 show pain data from female or male KL subgroups.
Tables 19-20 show pain data from KL subgroups subjects divided into BMI's.
Tables 21-31 show pain data from subjects divided into age groups.
Tables 32 show pain data from from KL subgroups subjects divided into ages less than or more than 70 years old.
Tables 33-34 show data on subjects treated with either PAAG-OA or Synvisc-One (hyalonic acid)
Table 35 show pain data from overweight subjects less than 70 years old.
Table 36 show pain data from overweight subjects more than 70 years old.
Table 37 show pain data from male subjects more than 70 years old.
Table 38 show pain data from overweight male subjects.
   An excerpt of data relating to ITT is shown in the following:

TABLE 5
Baseline characteristics (ITT)

| | PAAG-OA | Synvisc-One |
|---|---|---|
| ITT Analysis Set (N) | 119 | 120 |
| Age (years) | | |
| Median (min-max) | 67.0 (42-90) | 68.5 (31-85) |
| Age group (N, %) | | |
| <50 years | 8 (6.7) | 6 (5.0) |
| 50-60 years | 15 (12.6) | 20 (16.7) |
| 60-70 years | 47 (39.5) | 42 (35.0) |
| ≥70 years | 49 (41.2) | 52 (43.3) |
| Sex (N, %) | | |
| Female | 58 (48.7) | 68 (56.7) |
| Male | 61 (51.3) | 52 (43.3) |
| Race (N, %) | | |
| Multiple¹⁾ | 0 | 1 (0.8) |
| White | 118 (99.2) | 119 (99.2) |
| Other²⁾ | 1 (0.8) | 0 |
| Height (cm) | | |
| Median (min-max) | 173.0 (155-201) | 172.5 (152-195) |
| Weight (kg) | | |
| Median (min-max) | 81.0 (53-129) | 80.0 (55-113) |
| BMI (kg/m²) | | |
| Median (min-max) | 27.2 (20.4-35.0) | 26.8 (20.0-34.9) |
| BMI group (N, %) | | |
| Normal³⁾ | 26 (21.8) | 39 (32.5) |
| Overweight⁴⁾ | 63 (52.9) | 53 (44.2) |
| Obese⁵⁾ | 30 (25.2) | 28 (23.3) |
| Time since OA | | |
| diagnosis (years) | | |
| Median (min-max) | 7.5 (0.0-42.0) | 6.0 (0.0-41) |

¹⁾White/Mother Russian, Father Iranian;
²⁾Father Chinese, Mother Danish;
³⁾Normal: 18.5-24.9 kg/m²;
⁴⁾Overweight: 25-29.9 kg/m²;
⁵⁾Obese: ≥30 kg/m²

TABLE 6
Age (years) by KL grade-ITT

| | PAAG-OA | Synvisc-One |
|---|---|---|
| ITT Analysis Set (N) | 119 | 120 |
| KL grade 2 | | |
| Median (min-max) | 67 (46-88) | 67 (31-85) |
| KL grade 3 | | |
| Median (min-max) | 67 (42-79) | 70 (55-84) |
| KL grade 4 | | |
| Median (min-max) | 73 (67-90) | 70 (49-80) |

TABLE 7
Selected demographics for normal weight subjects-ITT

| | PAAG-OA | Synvisc-One |
|---|---|---|
| ITT Analysis Set (N) | 26 | 39 |
| Age (years) | | |
| Median (min-max) | 65 (42-80) | 67 (42-85) |
| Sex (N, %) | | |
| Female | 12 (46.2) | 26 (66.7) |
| Male | 14 (53.8) | 13 (33.3) |
| Time since OA | | |
| diagnosis (years) | | |
| Median (min-max) | 6.5 (1.0-42.0) | 8 (0.0-30.0) |
| KL grade | | |
| Grade 2 | 15 (57.7) | 22 (56.4) |
| Grade 3 | 9 (34.6) | 13 (3.3) |
| Grade 4 | 2 (7.7) | 4 (10.3) |

### Reduction of WOMAC Pain-Total KL Grade 2-4 Subjects-Tables 8-10

Aim of this study was to show WOMAC pain in the group total KL grade 2-4.

In the following tables data relating to the WOMAC pain subscale is presented: As mentioned previously, the WOMAC consists of three subscales:
pain (five questions)
stiffness (two questions)
physical function (17 questions).

The subscale scores can vary, with pain ranging from 0 to 20 points; stiffness, 0 to 8 points; and physical function, 0 to 68 points. Accordingly, higher scores indicate worse pain, stiffness, and functional limitations.

TABLE 8
Post Hoc: Total KL grade 2-4 subjects with at least
50% reduction in WOMAC pain subscale-ITT

| | PAAG-OA |
|---|---|
| | (n/N (%)) |
| ITT analysis set (N) | 119 |
| Week 26 (½ year) | |
| Non-missing pain subscale | 114 |
| Reduction in pain subscale | 90/114 (78.9) |
| At least 50% reduction in pain* | 48/90 (53.3) |
| At least 50% reduction in transformed | 48/114 (42.1) |
| WOMAC pain subscale (0-100) | |
| Week 52 (1 year) | |
| Non-missing pain subscale | 107 |
| Reduction in pain subscale | 78/107 (72.9) |
| At least 50% reduction in pain* | 44/78 (56.4) |
| At least 50% reduction in transformed | 44/107 (41.1) |
| WOMAC pain subscale (0-100) | |

*At least 50% reduction is relative to number of subjects with a reduction
P-value comes from a Fisher′s exact test for independence

### Conclusion Table 8

Table 8 demonstrates that after ½ year, about 42% of KL grade 2-4 subjects experience at least 50% reduction in transformed WOMAC pain subscale (0-100. This effect is maintained at 41% after 1 year, and increases to about 48% two years after treatment (see Table 11B).

Accordingly, between 42-48% of the total of KL grade 2-4 subjects show at least 50% reduction in transformed WOMAC pain subscale (0-100) within a span of 2 years after treatment. Thus, a clear reduction in the pain in total of KL grade 2-4 subjects is observed.

TABLE 9
Post Hoc: Total KL grade 2-4 subjects with at least
12 points reduction in WOMAC pain subscale-ITT

| | PAAG-OA |
|---|---|
| | n/N (%) |
| ITT analysis set (N) | 119 |
| Week 26 (½ year) | |
| Non-missing pain subscale | 114 |
| Reduction in pain subscale | 90/114 (78.9) |
| At least 12 points reduction in pain* | 63/90 (70.0) |
| At least 12 points reduction in transformed | 63/114 (55.3) |
| WOMAC pain subscale (0-100) | |
| Week 52 (1 year) | |
| Non-missing pain subscale | 107 |
| Reduction in pain subscale | 78/107 (72.9) |
| At least 12 points reduction in pain* | 58/78 (74.4) |
| At least 12 points reduction in transformed | 58/107 (54.2) |
| WOMAC pain subscale (0-100) | |

*At least 12 points reduction is relative to number of subjects with a reduction
P-value comes from a Fisher′s exact test for independence

### Conclusion Table 9

Table 9 demonstrates that after ½ year, about 55% of KL grade 2-4 subjects experience at least 12 points reduction in transformed WOMAC pain subscale (0-100. This effect is maintained at 54% after 1 year, but increases to about 59% two years after treatment (see Table 12B).

Accordingly, between 55-59% of the total of KL grade 2-4 subjects show at least 12 points reduction in transformed WOMAC pain subscale (0-100) within a span of 2 years after treatment. Thus, a clear reduction in the pain in total of KL grade 2-4 subjects is observed.

TABLE 10
Post Hoc: Total KL grade 2-4 subjects with at least
20 points reduction in WOMAC-pain subscale-ITT

| | PAAG-OA |
|---|---|
| | n/N (%) |
| ITT analysis set (N) | 119 |
| Week 26 (½ year) | |
| Non-missing pain subscale | 114 |
| Reduction in pain subscale | 90/114 (78.9) |
| At least 20 points reduction in pain* | 56/90 (62.2) |
| At least 20 points reduction in transformed | 56/114 (49.1) |
| WOMAC pain subscale (0-100) | |
| Week 52 (1 year) | |
| Non-missing pain subscale | 107 |
| Reduction in pain subscale | 78/107 (72.9) |
| At least 20 points reduction in pain* | 45/78 (57.7) |
| At least 20 points reduction in transformed | 45/107 (42.1) |
| WOMAC pain subscale (0-100) | |

*At least 20 points reduction is relative to number of subjects with a reduction
P-value comes from a Fisher′s exact test for independence

### Conclusion Table 10

Table 10 demonstrates that after ½ year, about 49% of KL grade 2-4 subjects experience at least 20 points reduction in transformed WOMAC pain subscale (0-100. This effect is reduced to 42% at 1 year, but increases again to 49% at two years (see Table 13B).

Accordingly, between 42-49% of the total of KL grade 2-4 subjects show at least points reduction in transformed WOMAC pain subscale (0-100) within a span of 2 years after treatment. Thus, a clear reduction in the pain in total of KL grade 2-4 subjects is observed.

### Summary Conclusion Tables 8-10

Tables 8-10 demonstrates that a clear reduction in the pain in total of KL grade 2-4 subjects is observed even after 52 weeks, and even more so after 104 weeks (Tables 11B, 12B and 13B).

### Reduction of WOMAC Pain-KL Grade 2, 3, 4, 2-3 and 2-4

### Aim of Study

Aim of study was to investigate WOMAC pain effect in subgroups of KL grade 2, 3, 4, 2-3 and 2-4.

The following tables 11-14 present the results of pain after treatment of PAAG in KL subgroups.

TABLE 11A
Subjects with at least 50% reduction in transformed WOMAC pain
subscale-subjects with KL grade 2, 3 and 4-blinded phase-ITT analysis set

| | KL grade 2 | KL grade 3 | KL grade 4 |
|---|---|---|---|
| | N (%) | N (%) | N (%) |
| ITT analysis set (N) | 67 | 39 | 13 |
| Week 26 (½ year) | | | |
| Non-missing WOMAC pain | 63 | 38 | 13 |
| subscale (N) | | | |
| Reduction in pain subscale | 50/63 (79.4) | 29/38 (76.3) | 11/13 (84.6) |
| At least 50% reduction in | 27/50 (54.0) | 15/29 (51.7) | 6/11(54.5) |
| pain* | | | |
| At least 50% reduction in | 27/63 (42.9) | 15/38 (39.5) | 6/13 (46.2) |
| pain | | | |
| Week 52 (1 year) | | | |
| Non-missing WOMAC pain | 61 | 35 | 11 |
| subscale (N) | | | |
| Reduction in pain subscale | 47/61 (77.0) | 25/35 (71.4) | 6/11 (54.5) |
| At least 50% reduction in | 27/47 (57.4) | 14/25 (56.0) | 3/6 (50.0) |
| pain* | | | |
| At least 50% reduction in | 27/61 (44.3) | 14/35 (40.0) | 3/11 (27.3) |
| pain | | | |

N: Number of subjects,
%: Percentage of subjects
At least 50% reduction is relative to number of subjects with an actual reduction

P-Values Come from Fisher's Exact Tests for Independence

TABLE 11B
Summary of subjects with at least 50% reduction in transformed WOMAC pain subscale
(0-100)-subjects with KL grade 2, 3, 4, 2-3 and 2-4-blinded phase-ITT analysis set.

| | | | | | TOTAL |
|---|---|---|---|---|---|
| | KL grade 2 | KL grade 3 | KL grade 4 | KL grade 2-3 | (KL grade 2-4) |
| | n/N (%) | n/N (%) | n/N (%) | n/N (%) | n/N (%) |
| Week 26 (½ year) | | | | | |
| At least 50% | 27/63 | 15/38 | 6/13 | 42/101 | 48/114 |
| reduction in pain | (42.9%) | (39.5%) | (46.2%) | (41.6%) | (42.1%) |
| Week 52 (1 year) | | | | | |
| At least 50% | 27/61 | 14/35 | 3/11 | 41/96 | 44/107 |
| reduction in pain | (44.3%) | (40.0%) | (27.3%) | (42.7%) | (41.1%) |
| Week 104 (2 years) | | | | | |
| At least 50% | 26/47 | 14/32 | 2/8 | 40/79 | 42/87 |
| reduction in pain | (55.3%) | (43.8%) | (25.0%) | (50.6%) | (48.3%) |

N: Number of subjects,
%: Percentage of subjects

### Conclusion Tables 11A and 11B

The summary results presented in table 11A are included in overview Table 11B. Table 11B also include data from the KL groups 2-3 and 2-4.

Table 11B clearly demonstrate that after 1 year ca. 40-44% of KL grade 2 and/or KL grade 3 subjects experience at least 50% reduction in transformed WOMAC pain subscale (0-100) compared to the subgroup of KL grade 4 patients, wherein about 27% of the subjects experience at least 50% reduction in transformed WOMAC pain subscale (0-100).

An even higher percentage of subjects that experience an at least 50% reduction in transformed WOMAC pain subscale (0-100) is observed after 2 years, where ca. 44-55% of subjects of KL grade 2 and/or KL grade 3 subjects experience at least 50% reduction in transformed WOMAC pain subscale (0-100) compared to the subgroup of KL grade 4 patients, for which about 25% of the subjects experience at least 50% reduction in transformed WOMAC pain subscale (0-100).

The results presented in Table 11B also demonstrate that a higher percentage of KL grade 2 subjects experience pain relief compared to KL grade 3 subjects. This is evident after ½, 1 and 2 years of treatment.

Thus, a clear reduction in the pain in total of KL grade 2-3 subjects is observed.

TABLE 12A
Subjects with at least 12 points reduction in transformed WOMAC pain subscale (0-100)-subjects
with KL grade 2, 3 and 4-blinded phase-ITT analysis set.

| | KL grade 2 | KL grade 3 | KL grade 4 |
|---|---|---|---|
| | N (%) | N (%) | N (%) |
| ITT analysis set (N) | 67 | 39 | 13 |
| Week 26 | | | |
| Non-missing WOMAC pain | 63 | 38 | 13 |
| subscale (N) | | | |
| Reduction in pain subscale (N) | 50/63 (79.4) | 29/38 (76.3) | 11/13 (84.6) |
| At least 12 point reduction in pain* | 35/50 (70.0) | 23/29 (79.3) | 5/11 (45.5) |
| At least 12 point reduction in pain | 35/63 (55.6) | 23/38 (60.5) | 5/13 (38.5) |
| Week 52 | | | |
| Non-missing WOMAC pain | 61 | 35 | 11 |
| subscale (N) | | | |
| Reduction in pain subscale (N) | 47/61 (77.0) | 25/35 (71.4) | 6/11 (54.5) |
| At least 12 point reduction in pain* | 36/47 (76.6) | 18/25 (72.0) | 4/6 (66.7) |
| At least 12 point reduction in pain | 36/61 (59.0) | 18/35 (51.4) | 4/11 (36.4) |

N: Number of subjects,
%: Percentage of subjects
*At least 12 point reduction is relative to number of subjects with an actual reduction
P-values come from Fisher′s exact tests for independence

TABLE 12B
Summary of subjects with at least 12 points reduction in
transformed WOMAC pain subscale (0-100) - subjects with KL
grade 2, 3, 4, 2-3 and 2-4-blinded phase - ITT analysis set.

| | | | | |
|---|---|---|---|---|
| | | | KL | TOTAL |
| KL | KL | KL | grade | (KL grade |
| grade 2 | grade 3 | grade 4 | 2-3 | 2-4) |
| n/N (%) | n/N (%) | n/N (%) | n/N (%) | n/N (%) |

Week 26 (½ year)

| | | | | | |
|---|---|---|---|---|---|
| At least | 35/63 | 23/38 | 5/13 | 58/101 | 63/114 |
| 12 point | (55.6%) | (60.5%) | (38.5%) | (57.4%) | (55.3%) |
| reduction | | | | | |
| in pain | | | | | |

Week 52 (1 year)

| | | | | | |
|---|---|---|---|---|---|
| At least | 36/61 | 18/35 | 4/11 | 54/96 | 58/107 |
| 12 point | (59.0%) | (51.4%) | (36.4%) | (56.3%) | (54.2%) |
| reduction | | | | | |
| in pain | | | | | |

Week 104 (2 years)

| | | | | | |
|---|---|---|---|---|---|
| At least | 28/47 | 19/32 | 4/8 | 47/79 | 51/87 |
| 12 point | (59.6%) | (59.4%) | (50.0%) | (59.5%) | (58.6%) |
| reduction | | | | | |
| in pain | | | | | |

N: Number of subjects,
%: Percentage of subjects

Conclusion tables 12A and 12B

The summary results presented in table 12A are included in overview Table 12B. Table 12B clearly demonstrate that after ½, 1 and 2 years, a higher percentage of subjects from the KL grade 2 and/or KL grade 3 groups experience at least 12 points reduction in transformed WOMAC pain subscale (0-100) compared to the subgroup of KL grade 4 patients. The improvement is equally pronounced for the KL group 2 and 3 subjects after 2 year.

Thus, a clear and consistent improvement in the reduction in the pain is seen for KL group 2 and/or 3 subjects compared to KL group 4 subjects after at least 12, 1 and 2 years.

TABLE 13A
Subjects with at least 20 points reduction in transformed
WOMAC pain subscale (0-100) - subjects with KL grade 2, 3 and
4 - blinded phase - ITT analysis set.

| | KL grade 2 | KL grade 3 | KL grade 4 |
|---|---|---|---|
| | N (%) | N (%) | N (%) |
| ITT analysis set (N) | 67 | 39 | 13 |

Week 26

| | | | |
|---|---|---|---|
| Non-missing WOMAC | 63 | 38 | 13 |
| pain subscale (N) | | | |
| Reduction in pain | 50/63 ( 79.4) | 29/38 ( 76.3) | 11/13 ( 84.6) |
| subscale (N) | | | |
| At least 20 point | 29/50 ( 58.0) | 22/29 ( 75.9) | 5/11 ( 45.5) |
| reduction in pain* | | | |
| At least 20 point | 29/63 ( 46.0) | 22/38 ( 57.9) | 5/13 ( 38.5) |
| reduction in pain | | | |

Week 52

| | | | |
|---|---|---|---|
| Non-missing WOMAC | 61 | 35 | 11 |
| pain subscale (N) | | | |
| Reduction in pain | 47/61 ( 77.0) | 25/35 ( 71.4) | 6/11 ( 54.5) |
| subscale (N) | | | |
| At least 20 point | 29/47 ( 61.7) | 13/25 ( 52.0) | 3/6 ( 50.0) |
| reduction in pain* | | | |
| At least 20 point | 29/61 ( 47.5) | 13/35 ( 37.1) | 3/11 ( 27.3) |
| reduction in pain | | | |

N: Number of subjects,
%: Percentage of subjects
*At least 20 point reduction is relative to number of subjects with an actual reduction.
P-values come from Fisher's exact tests for independence

TABLE 13B
Subjects with at least 20 points reduction in transformed
WOMAC pain subscale (0-100) - subjects with KL grade 2, 3, 4,
2-3 and 2-4 - blinded phase - ITT analysis set.

| | | | | |
|---|---|---|---|---|
| | | | | TOTAL |
| KL grade | KL grade | KL grade | KL grade | (KL grade |
| 2 | 3 | 4 | 2-3 | 2-4) |
| n/N (%) | n/N (%) | n/N (%) | n/N (%) | n/N (%) |

Week 26 (½ year)

| | | | | | |
|---|---|---|---|---|---|
| At least | 29/63 | 22/38 | 5/13 | 51/101 | 56/114 |
| 20 point | (46.0%) | (57.9%) | (38.5%) | (50.5%) | (49.1%) |
| reduction | | | | | |
| in pain | | | | | |

Week 52 (1 year)

| | | | | | |
|---|---|---|---|---|---|
| At least | 29/61 | 13/35 | 3/11 | 42/96 | 45/107 |
| 20 point | (47.5%) | (37.1%) | (27.3%) | (43.8%) | (42.1%) |
| reduction | | | | | |
| in pain | | | | | |

Week 104 (2 years)

| | | | | | |
|---|---|---|---|---|---|
| At least | 26/47 | 14/32 | 3/8 | 40/79 | 43/87 |
| 20 point | (55.3%) | (43.8%) | (37.5%) | (50.6%) | (49.4%) |
| reduction | | | | | |
| in pain | | | | | |

N: Number of subjects,
%: Percentage of subjects

### Conclusion Tables 13A and 13B

The summary results presented in table 13A are included in overview Table 13B. Table 13B clearly demonstrate that after ½, 1 and 2 years, a higher percentage of subjects from the KL grade 2 and/or KL grade 3 groups experience at least 20 points reduction in transformed WOMAC pain subscale (0-100) compared to the subgroup of KL grade 4 subjects. The improvement is most pronounced for the KL group 2 subjects after 1 and 2 years for both the KL grade 2 and/or KL grade 3 subgroups.

Thus, a clear improvement in the reduction in the pain is seen for KL group 2 and/or 3 subjects compared to KL group 4 subjects after at least ½, 1 and 2 years.

Conclusion on Pain Data from Tables 11-13

As demonstrated by tables 11-13, an increased number of subjects diagnosed with KL grade 2 and/or KL grade 3 experience less pain as compared to subjects diagnosed with KL grades 4 after treatment with PAAG. This pain reducing effect is observed with all three WOMAC pain parameters. The surprisingly good results are sustained for at least 2 years, but also show impressive pain reducing effect after at least ½ and at least 1 year.

Reduction of WOMAC Pain-KL Grade 2, 3, 4-Baseline, Week 4, 12, 26 and 52-Detailed Values and Change from Baseline.

Tables 14 and 15 depicts further details of the data from the clinical study.

TABLE 14
Summary of transformed WOMAC pain subscale (0-100) by Kellgren-
Lawrence grade at baseline-blinded phase-ITT analysis set

| | KL Grade 2 | | | | KL Grade 3 | | | | KL Grade 4 | | | |
|---|---|---|---|---|---|---|---|---|---|---|---|---|
| | | | Change | | | | Change | | | | Change | |
| | Actual | | from | | Actual | | from | | Actual | | from | |
| | values | | baseline | | values | | baseline | | values | | baseline | |
| ITT analysis | 67 | (100.0) | | | 39 | (100.0) | | | 13 | (100.0) | | |
| set (N,%) | | | | | | | | | | | | |
| Baseline | | | | | | | | | | | | |
| N | 67 | | | | 39 | | | | 13 | | | |
| Mean (SD) | 44.3 | (13.2) | | | 47.6 | (13.5) | | | 41.9 | (14.2) | | |
| Median | 45.0 | | | | 45.0 | | | | 45.0 | | | |
| Min-Max | 10-75 | | | | 25-70 | | | | 20-60 | | | |
| Week 4 | | | | | | | | | | | | |
| N | 67 | | 67 | | 39 | | 39 | | 13 | | 13 | |
| Mean (SD) | 34.5 | (18.8) | −9.8 | (19.8) | 29.6 | (16.1) | −17.9 | (16.0) | 31.9 | (20.0) | −10.0 | (18.8) |
| Median | 30.0 | | −10.0 | | 30.0 | | −15.0 | | 30.0 | | −10.0 | |
| Min-Max | 0-100 | | −65-75 | | 0-70 | | −65-25 | | 0-55 | | −50-20 | |
| Week 12 | | | | | | | | | | | | |
| N | 65 | | 65 | | 37 | | 37 | | 13 | | 13 | |
| Mean (SD) | 26.1 | (17.8) | −18.8 | (18.0) | 26.5 | (15.5) | −20.8 | (21.6) | 23.1 | (14.9) | −18.8 | (18.9) |
| Median | 25.0 | | −15.0 | | 25.0 | | −25.0 | | 15.0 | | −20.0 | |
| Min-Max | 0-65 | | −60-25 | | 0-60 | | −65-25 | | 5-55 | | −45-10 | |
| Week 26 | | | | | | | | | | | | |
| N | 63 | | 63 | | 38 | | 38 | | 13 | | 13 | |
| Mean (SD) | 27.2 | (19.0) | −17.3 | (18.8) | 26.2 | (16.8) | −21.2 | (20.7) | 26.2 | (16.5) | −15.8 | (14.7) |
| Median | 25.0 | | −15.0 | | 25.0 | | −20.0 | | 20.0 | | −10.0 | |
| Min-Max | 0-75 | | −65-20 | | 0-75 | | −65-10 | | 0-60 | | −40-5 | |
| Week 52 | | | | | | | | | | | | |
| N | 61 | | 61 | | 35 | | 35 | | 11 | | 11 | |
| Mean (SD) | 25.0 | (20.5) | −18.7 | (20.5) | 26.7 | (18.5) | −20.1 | (24.0) | 31.8 | (17.6) | −9.1 | (16.6) |

| | | | | | | |
|---|---|---|---|---|---|---|
| Median | 20.0 | −15.0 | 35.0 | −15.0 | 30.0 | −10.0 |
| Min-Max | 0-75 | −60-20 | 0-65 | −65-10 | 10-65 | −40-15 |

N: Number of subjects,
SD: Standard deviation

TABLE 15
Analysis of change from baseline to week 52 in transformed
WOMAC pain subscale (0-100) by Kellgren-Lawrence grade
at baseline-blinded phase - ITT analysis set.

| | KL grade 2 | | KL grade 3 | | KL grade 4 | |
|---|---|---|---|---|---|---|
| | | LSMean | | LSMean | | LSMean |
| | N | (95% CI) | N | (95% CI) | N | (95% CI) |
| Week 4 | | | | | | |
| PAAG-OA | 67 | −11.82 | 39 | −17.00 | 13 | −9.42 |
| | | (−15.84; −7.80) | | (−21.86; −12.15) | | (−19.55; 0.71) |
| Week 13 | | | | | | |
| PAAG-OA | 65 | −19.86 | 37 | −18.98 | 13 | −17.82 |
| | | (−23.89; −15.84) | | (−24.15; −13.82) | | (−25.91; −9.72) |
| Week 26 | | | | | | |
| PAAG-OA | 63 | −18.07 | 38 | −20.38 | 13 | −15.25 |
| | | (−22.55; −13.59) | | (−26.02; −14.73) | | (−24.17; −6.33) |
| Week 52 | | | | | | |
| PAAG-OA | 61 | −18.79 | 35 | −18.39 | 11 | −10.30 |
| | | (−23.51; −14.07) | | (−24.27; −12.51) | | (−20.61; 0.01) |

N: Number of subjects contributing to the analysis.
The analysis is performed on change from baseline using a mixed model for repeated measures including fixed, categorical effects of treatment, week, treatment-by-week interaction and site, as well as the baseline value and baseline-by-week interaction as covariates.

### Conclusion

Tables 14-15 demonstrate that treatment with PAAG in subjects diagnosed with OA KL grade 2 or 3 experiences an increased reduction in pain as compared to subjects diagnosed with KL grade 4. This is particularly observed after week 26, and even more so after week 52.

### Stiffness and Physical Function:

### Aim of Study

To evaluate the WOMAC pain subscales “physical function” and “stiffness” in KL grade subgroups KL grade 2, 3, 4, 2-3 and 2-4.

It should be noted that subjects experiencing a better physical function or less stiffness show this by obtaining lower mean values.

TABEL 16
Subjects results in relation to the WOMAC pain subscale “physical
function” - subjects with KL grade 2, 3, 4, 2-3 and 2-4 - blinded
phase - ITT analysis set.
WOMAC physical function

| | | | | TOTAL |
|---|---|---|---|---|
| KL grade | KL grade | KL grade | KL grade | (KL grade |
| 2 | 3 | 4 | 2-3 | 2-4) |

Week 26 (½ year)

| | | | | | |
|---|---|---|---|---|---|
| N | 63 | 38 | 13 | 101 | 114 |
| Mean | −19.0 | −21.1 | −20.5 | −19.8 | −19.9 |
| (SD) | (20.6) | (22.4) | (13.9) | (21.2) | (20.4) |

Week 52 (1 year)

| | | | | | |
|---|---|---|---|---|---|
| N | 61 | 35 | 11 | 96 | 107 |
| Mean | −19.7 | −21.0 | −11.4 | −20.2 | −19.3 |
| (SD) | (21.8) | (23.1) | (14.8) | (22.2) | (21.7) |

Week 104 (2 years)

| | | | | | |
|---|---|---|---|---|---|
| N | 47 | 32 | 8 | 79 | 87 |
| Mean | −20.8 | −18.4 | −10.5 | −19.8 | −19.0 |
| (SD) | (19.1) | (21.8) | (12.6) | (20.2) | (19.7) |

N: Number of subjects,
SD: Standard deviation.
Higher scores indicate worse functional limitations.

TABEL 17
Subjects results in relation to the WOMAC pain subscale
“stiffness” - subjects with KL grade 2, 3, 4, 2-3 and 2-4 - blinded
phase - ITT analysis set.
WOMAC stiffness subscale

| | | | | TOTAL |
|---|---|---|---|---|
| KL grade | KL grade | KL grade | KL grade | (KL grade |
| 2 | 3 | 4 | 2-3 | 2-4) |

Week 26 (½ year)

| | | | | | |
|---|---|---|---|---|---|
| N | 63 | 38 | 13 | 101 | 114 |
| Mean | −19.0 | −21.1 | −18.3 | −19.8 (26.9) | −19.6 (26.3) |
| (SD) | (26.3) | (28.2) | (22.6) | | |

Week 52 (1 year)

| | | | | | |
|---|---|---|---|---|---|
| N | 61 | 35 | 11 | 96 | 107 |
| Mean | −21.5 | −22.1 | −1.1 (27.1) | −21.7 (27.2) | −19.6 (27.7) |
| (SD) | (27.2) | (27.5) | | | |

Week 104 (2 years)

| | | | | | |
|---|---|---|---|---|---|
| N | 47 | 32 | 8 | 79 | 87 |
| Mean | −20.7 | −18.8 | −15.6 | −19.9 (26.1) | −19.5 (25.1) |
| (SD) | (25.8) | (26.8) | (11.1) | | |

N: Number of subjects,
SD: Standard deviation.
Higher scores indicate worse stiffness.

### Results of Tables 16 and 17:

Physical function: The results of table 16 clearly show that KL grade 2, KL grade 3 and group KL grade 2-3 subjects experience a better physical function (lower mean values) than KL grade 4 subjects do at timepoints 1 and 2 years. Additionally, and surprisingly, KL grade 3 subjects also experience a small improvement after only ½ year compared to KL grade 4.

Stiffness: The results of table 17 clearly show that KL grade 2, KL grade 3 and group KL grade 2-3 subjects experience less stiffness (lower values) than KL grade 4 patients do at timepoints ½, 1 and 2 years.

### Gender

### Aim of Study:

Aim of study was to investigate the WOMAC pain effect in female or male subgroups of KL grade 2, 3, 4, 2-3 and 2-4.

### Results—Gender

The following tables 18A-18B present the results of pain measurements after treatment of PAAG in subjects with a certain age in various KL subgroups.

TABLE 18A
Post Hoc: Analysis of change from baseline to week 26 in
transformed WOMAC pain subscale by gender - ITT.

| | Week 26 | | Week 52 | |
|---|---|---|---|---|
| | N | LSMeans (95% CI) | N | LSMeans (95% CI) |
| Female | | | | |
| PAAG-OA | 55 | −18.48 (−23.35; −13.60) | 51 | −16.55 (−21.67; −11.43) |
| Male | | | | |
| PAAG-OA | 59 | −18.70 (−22.81; −14.59) | 56 | −19.41 (−23.90; −14.92) |

N: Number of subjects contributing to the analysis.
The analysis is performed on change from baseline using a mixed model for repeated measures including fixed, categorical effects of treatment, week, treatment-by-week interaction and site, as well as the baseline value and baseline-by-week interaction as covariates.

TABLE 18B
Female subjects with at least 50% reduction in transformed
WOMAC pain subscale - or at least 12 or 20 point reduction in
pain - female subjects with KL grade 2, 3, 2-3, 4 and 2-4 -
blinded phase - ITT analysis set

| | | | | | |
|---|---|---|---|---|---|
| | | | | | TOTAL |
| | KL | KL | KL | KL grade | (KL grade |
| | grade 2 | grade 3 | grade 4 | 2-3 | 2-4) |
| Females | n/N (%) | n/N (%) | n/N (%) | n/N (%) | n/N (%) |

Week 26 (½ year)

| | | | | | |
|---|---|---|---|---|---|
| At least 50 % | 15/31 | 9/21 | 2/3 | 24/52 | 26/55 |
| reduction in pain | (48.4%) | (42.9%) | (66.7%) | (46.9%) | (47.3%) |
| At least 12 point | 20/31 | 13/21 | 1/3 | 33/52 | 34/55 |
| reduction in pain | (64.5%) | (61.9%) | (33.3%) | (63.5%) | (61.8%) |
| At least 20 point | 16/31 | 12/21 | 1/3 | 28/52 | 29/55 |
| reduction in pain | (51.6%) | (57.1%) | (33.3%) | (53.8%) | (52.7%) |

Week 52 (1 year)

| | | | | | |
|---|---|---|---|---|---|
| At least 50 % | 13/30 | 7/19 | 0/2 | 20/49 | 20/51 |
| reduction in pain | (43.3%) | (36.8%) | (0.0%) | (40.8%) | (39.2%) |
| At least 12 point | 15/30 | 9/19 | 0/2 | 24/49 | 24/51 |
| reduction in pain | (50.0%) | (47.4%) | (00.0%) | (49.0%) | (47.1%) |
| At least 20 point | 14/30 | 7/19 | 0/2 | 21/49 | 21/51 |
| reduction in pain | (46.7%) | (36.8%) | (00.0%) | (42.9%) | (41.2%) |

Week 104 (2 years)

| | | | | | |
|---|---|---|---|---|---|
| At least 50 % | 12/23 | 7/18 | 0/2 | 19/41 | 19/43 |
| reduction in pain | (52.2%) | (38.9%) | (0.0%) | (46.3%) | (44.2%) |
| At least 12 point | 11/23 | 10/18 | 0/2 | 21/41 | 21/43 |
| reduction in pain | (47.8%) | (55.6%) | (00.0%) | (51.2%) | (48.8%) |
| At least 20 point | 10/23 | 8/18 | 0/2 | 18/41 | 18/43 |
| reduction in pain | (43.5%) | (44.4%) | (00.0%) | (43.9%) | (41.9%) |

TABLE 18C
Male subjects with at least 50% reduction in transformed
WOMAC pain subscale - or at least 12 or 20 point reduction in
pain - male subjects with KL grade 2, 3, 2-3, 4 and 2-4 -
blinded phase - ITT analysis set

| | | | | | |
|---|---|---|---|---|---|
| | | | | | TOTAL |
| | KL | KL | KL | KL grade | (KL grade |
| | grade 2 | grade 3 | grade 4 | 2-3 | 2-4) |
| Males | n/N (%) | n/N (%) | n/N (%) | n/N (%) | n/N (%) |

Week 26 (½ year)

| | | | | | |
|---|---|---|---|---|---|
| At least 50 % | 12/32 | 6/17 | 4/10 | 18/49 | 22/59 |
| reduction in pain | (37.5%) | (35.3%) | (40.0%) | (36.7%) | (37.3%) |
| At least 12 point | 15/32 | 10/17 | 4/10 | 25/49 | 29/59 |
| reduction in pain | (46.9%) | (58.8%) | (40.0%) | (51.0%) | (49.2%) |
| At least 20 point | 13/32 | 10/17 | 4/10 | 23/49 | 27/59 |
| reduction in pain | (40.6%) | (58.8%) | (40.0%) | (46.9%) | (45.8%) |

Week 52 (1 year)

| | | | | | |
|---|---|---|---|---|---|
| At least 50 % | 14/31 | 7/16 | 3/9 | 21/47 | 24/56 |
| reduction in pain | (45.2%) | (43.8%) | (33.3%) | (44.7%) | (42.9%) |
| At least 12 point | 21/31 | 9/16 | 4/9 | 30/47 | 34/56 |
| reduction in pain | (67.7%) | (56.3%) | (44.4%) | (63.8%) | (60.7%) |
| At least 20 point | 15/31 | 6/16 | 3/9 | 21/47 | 24/56 |
| reduction in pain | (48.4%) | (37.5%) | (33.3%) | (44.7%) | (42.9%) |

Week 104 (2 years)

| | | | | | |
|---|---|---|---|---|---|
| At least 50 % | 14/24 | 7/14 | 2/6 | 21/38 | 23/44 |
| reduction in pain | (58.3%) | (50.0%) | (33.3%) | (55.3%) | (52.3%) |
| At least 12 point | 17/24 | 9/14 | 4/6 | 26/38 | 30/44 |
| reduction in pain | (70.8%) | (64.3%) | (66.7%) | (68.4%) | (68.2%) |
| At least 20 point | 16/24 | 6/14 | 3/6 | 22/38 | 25/44 |
| reduction in pain | (66.7%) | (42.9%) | (50.0%) | (57.9%) | (56.8%) |

### Conclusion—Gender—Tables 18A, 18B and 18C

Table 18A demonstrates that the treatment of OA with PAAG-OA is particularly useful for reduction of pain in males and females of KL grade 2-4.

Females: Table 18B surprisingly demonstrate that after 1 and 2 years, a larger percentage of female KL grade 2, 3 and 2-3 subjects experienced at least 50% reduction in transformed WOMAC pain subscale (0-100) compared to the subgroup of KL grade 4 subjects. Further, a larger percentage of female KL grade 2 subjects experienced at least 50% reduction in transformed WOMAC pain subscale (0-100) compared to the subgroup of KL grade 3 subjects after ½, 1 and 2 years.

These surprising effects were confirmed when the pain was measured with the percentage of subjects that experienced at least 12 or 20 point reduction in pain. In fact, after ½, 1 and 2 years, a larger percentage of female KL grade 2, 3 and 2-3 subjects experienced at least 12 or 20 point reduction in pain compared to the subgroup of KL grade 4 subjects.

Summary, females: The subgroups KL 2 and/or KL 3 and/or KL 2-3 for females subjects experienced a larger pain relief than the female KL 4 subgroup subjects.

Males: Table 18C surprisingly demonstrate that after 1 and 2 years, a larger percentage of male KL grade 2, 3 and/or KL grade 2-3 subjects experienced at least 50% reduction in transformed WOMAC pain subscale (0-100) compared to the subgroup of male KL grade 4 subjects. Further, a larger percentage of male KL grade 2 subjects experienced at least 50% reduction in transformed WOMAC pain subscale (0-100) compared to the subgroup of KL grade 3 subjects after ½, 1 and 2 years.

These surprising effects were confirmed when the pain was measured with the percentage of subjects that experienced at least 12 or 20 point reduction in pain for KL 2, KL 3 and KL 2-3 male subjects compared to KL 4 male subjects at 1 year and for KL 2 and KL 2-3 also at ½ and 2 years, except for KL 2 at ½ year for an at least 20 point reduction in pain, all when compared to KL 4 male subjects.

In addition after ½ and 1 years, a larger percentage of male KL grade 3 subjects experienced at least 12 or 20 point reduction in pain compared to the subgroup of KL grade 4 subjects.

Summary, males: The subgroup KL 2 and/or KL 3 and/or KL 2-3 for male subjects experienced a larger pain relief than the male KL 4 subgroup subjects.

### Results and Conclusion—Data on WOMAC Physical Function and Stiffness Pain Females and Males

Data on female WOMAC physical function pain parameters: Surprisingly, results on pain parameters from WOMAC physical function confirmed (data not shown) that female subgroups KL 2, KL 3, KL 2-3 and KL 2-4 experienced a larger pain relief than the female KL 4 subgroup subjects at timepoints ½, 1 and 2 years.

Data on female WOMAC stiffness pain parameters: Surprisingly, results on pain parameters from WOMAC stiffness confirmed (data not shown) that female subgroups KL 2, KL 3, KL 2-3 and KL2-4 experienced a larger pain relief than the female KL 4 subgroup subjects at timepoint 2 years.

Data on male WOMAC physical function pain parameters: Surprisingly, results on pain parameters from WOMAC physical function confirmed (data not shown) that male subgroups KL 2, KL 3, KL 2-3 and KL2-4 experienced a larger pain relief than the male KL 4 subgroup subjects at timepoints 1 and 2 years.

Data on male WOMAC stiffness pain parameters: Surprisingly, results on pain parameters from WOMAC stiffness confirmed (data not shown) that male subgroups KL 2, KL 3, KL 2-3 and KL2-4 experienced a larger pain relief than the male KL 4 subgroup subjects at timepoints ½, 1 and 2 years.

### BMI

### Aim of Study:

Aim of study was to investigate the WOMAC pain effect in subgroups of KL grade 2, 3, 4, 2-3 and 2-4 subjects having a BMI characterized as being either normal, overweight or obese.

### Results—BMI

The following tables 19-20 present the results of the BMI of subjects on pain after treatment of PAAG in KL subgroups.

TABLE 19
Summary of transformed WOMAC pain subscale (0-100) by BMI
group - blinded phase-ITT analysis set-Total KL grades 2-4.

| | Obese | | | | Overweight | | | | Normal | | | |
|---|---|---|---|---|---|---|---|---|---|---|---|---|
| | | | Change | | | | Change | | | | Change | |
| | Actual | | from | | Actual | | from | | Actual | | from | |
| | values | | baseline | | values | | baseline | | values | | baseline | |
| ITT | 30 | (100.0) | | | 63 | (100.0) | | | 26 | (100.0) | | |
| analysis set | | | | | | | | | | | | |
| (N, %) | | | | | | | | | | | | |
| Baseline | | | | | | | | | | | | |
| N | 30 | | | | 63 | | | | 26 | | | |
| Mean (SD) | 47.2 | (14.6) | | | 44.5 | (13.8) | | | 44.0 | (11.0) | | |
| Median | 47.5 | | | | 45.0 | | | | 45.0 | | | |
| Min-Max | 20-75 | | | | 10-70 | | | | 20-60 | | | |
| Week 4 | | | | | | | | | | | | |
| N | 30 | | 30 | | 63 | | 63 | | 26 | | 26 | |
| Mean (SD) | 35.8 | (21.2) | −11.3 | (21.5) | 31.8 | (18.4) | −12.7 | (19.1) | 30.8 | (12.7) | −13.3 | (14.8) |
| Median | 30.0 | | −12.5 | | 30.0 | | −10.0 | | 30.0 | | −10.0 | |
| Min-Max | 0-100 | | −50-75 | | 0-70 | | −65-35 | | 5-60 | | −55-25 | |
| Week 12 | | | | | | | | | | | | |
| N | 29 | | 29 | | 60 | | 60 | | 26 | | 26 | |
| Mean (SD) | 27.6 | (15.3) | −19.1 | (16.1) | 25.3 | (17.4) | −19.8 | (20.2) | 25.2 | (17.1) | −18.8 | (20.6) |
| Median | 25.0 | | −15.0 | | 25.0 | | −20.0 | | 25.0 | | −17.5 | |
| Min-Max | 0-60 | | −60-15 | | 0-65 | | −65-20 | | 0-60 | | −60-25 | |
| Week 26 | | | | | | | | | | | | |
| N | 29 | | 29 | | 61 | | 61 | | 24 | | 24 | |
| Mean (SD) | 28.8 | (18.5) | −19.1 | (17.7) | 26.4 | (18.9) | −18.0 | (21.6) | 25.2 | (14.8) | −18.5 | (13.0) |
| Median | 25.0 | | −20.0 | | 25.0 | | −15.0 | | 25.0 | | −20.0 | |
| Min-Max | 0-75 | | −50-15 | | 0-75 | | −65-20 | | 0-60 | | −40-5 | |
| Week 52 | | | | | | | | | | | | |
| N | 26 | | 26 | | 57 | | 57 | | 24 | | 24 | |
| Mean (SD) | 27.9 | (20.6) | −19.4 | (22.2) | 26.9 | (20.4) | −16.6 | (22.8) | 22.9 | (16.3) | −20.6 | (17.2) |

| | | | | | | |
|---|---|---|---|---|---|---|
| Median | 25.0 | −15.0 | 25.0 | −10.0 | 20.0 | −17.5 |
| Min-Max | 0-75 | −60-15 | 0-65 | −65-20 | 0-55 | −55-5 |

N: Number of subjects,
SD: Standard deviation

TABLE 20A
Analysis of change from baseline to week 52 in transformed WOMAC pain subscale
(0-100) by BMI group-blinded phase-ITT analysis set-Total KL grades 2-4.

| | Normal | | Overweight | | Obese | |
|---|---|---|---|---|---|---|
| | | LSMean (95% | | LSMean (95% | | LSMean (95% |
| | N | CI) | N | CI) | N | CI) |
| Week 4 | | | | | | |
| PAAG- | 26 | −13.47 | 63 | −13.53 | 30 | −11.66 |
| OA | | (−18.62; −8.31) | | (−17.60; −9.47) | | (−18.74; −4.59) |
| Week 13 | | | | | | |
| PAAG- | 26 | −19.07 | 60 | −19.75 | 29 | −19.79 |
| OA | | (−25.17; −12.97) | | (−23.86; −15.65) | | (−25.60; −13.97) |
| Week 26 | | | | | | |
| PAAG- | 24 | −18.97 | 61 | −18.48 | 29 | −18.16 |
| OA | | (−25.73; −12.21) | | (−22.97; −13.99) | | (−24.59; −11.73) |
| Week 52 | | | | | | |
| PAAG- | 24 | −21.41 | 57 | −16.14 | 26 | −17.79 |
| OA | | (−27.80; −15.02) | | (−20.97; −11.30) | | (−25.57; −10.02) |

N: Number of subjects contributing to the analysis.
The analysis is performed on change from baseline using a mixed model for repeated measures including fixed, categorical effects of treatment, week, treatment-by-week interaction and site, as well as the baseline value and baseline-by-week interaction as covariates.
BMI group:
Normal: 18.5-24.9 kg/m²,
Overweight: 25-29.9 kg/m²,
Obese: above 30 kg/m²

### Conclusion Tables 19 and 20A

Table 19 and 20A demonstrate that 52 weeks after treatment of a group of total KL grades 2-4 subjects, normal weight persons appears to have less pain than subjects with a BMI above 25. However, less pain is also observed for overweight and obese subjects.

TABLE 20B
Subjects categorized with BMIs being normal with at least 50%
reduction in transformed WOMAC pain subscale, or at least 12 or
20 points reduction in pain - subjects with KL grade 2, 3, 4, 2-3 and
2-4 - blinded phase - ITT analysis set

| | | | | | |
|---|---|---|---|---|---|
| | | | | | TOTAL |
| | KL | KL | KL | KL grade | (KL grade |
| | grade 2 | grade 3 | grade 4 | 2-3 | 2-4) |
| BMI normal* | n/N (%) | n/N (%) | n/N (%) | n/N (%) | n/N (%) |

Week 26 (½ year)

| | | | | | |
|---|---|---|---|---|---|
| At least 50 % | 4/14 | 5/8 | 1/2 | 9/22 | 10/24 |
| reduction in pain | (28.6%) | (62.5%) | (50.0%) | (40.9%) | (41.7%) |
| At least 12 point | 8/14 | 7/8 | 0/2 | 15/22 | 15/24 |
| reduction in pain | (57.1%) | (87.5%) | (00.0%) | (68.2%) | (62.5%) |
| At least 20 point | 6/14 | 7/8 | 0/2 | 13/22 | 13/24 |
| reduction in pain | (42.9%) | (87.5%) | (00.0%) | (59.1%) | (54.2%) |

Week 52 (1 year)

| | | | | | |
|---|---|---|---|---|---|
| At least 50 % | 7/15 | 4/7 | 0/2 | 11/22 | 11/24 |
| reduction in pain | (46.7%) | (57.1%) | (00.0%) | (50.0%) | (45.8%) |
| At least 12 point | 11/15 | 6/7 | 0/2 | 17/22 | 17/24 |
| reduction in pain | (73.3%) | (85.7%) | (00.0%) | (77.3%) | (70.8%) |
| At least 20 point | 9/15 | 3/7 | 0/2 | 12/22 | 12/24 |
| reduction in pain | (60.0%) | (42.9%) | (00.0%) | (54.5%) | (50.0%) |

Week 104 (2 years)

| | | | | | |
|---|---|---|---|---|---|
| At least 50 % | 8/13 | 4/6 | 0/2 | 12/19 | 12/21 |
| reduction in pain | (61.5%) | (66.7%) | (00.0%) | (63.2%) | (57.1%) |
| At least 12 point | 11/13 | 5/6 | 0/2 | 16/19 | 16/21 |
| reduction in pain | (84.6%) | (83.3%) | (00.0%) | (84.2%) | (76.2%) |
| At least 20 point | 10/13 | 4/6 | 0/2 | 14/19 | 14/21 |
| reduction in pain | (76.9%) | (66.7%) | (00.0%) | (73.7%) | (66.7%) |

*Major adult BMI classifications are underweight (under 18.5 kg/m2), normal weight (18.5 to 24.9), overweight (25 to 29.9), and obese (30 or more).

### Conclusion Table 20B: Normal BMI Subjects

Table 20B surprisingly show that at least 50% reduction in WOMAC pain is improved for normal BMI subjects for KL 2, KL 3 and KL 2-3 compared to the KL 4 subjects at time points 1 and 2 years and that this effect is increasing over time i.e. to be ca. 77%, 67% and 63% after 2 years of treatment. It should be noted that the number of subjects for the KL4 group is rather small and may be used to consider trends. Also, for KL3 normal weight subjects, the percentage of at least 50% reduction in WOMAC pain is improved over KL 4 subjects already at ½ year. Further, table 20B show that subjects experiencing at least 12 point reduction in pain is high when observed for normal weight subjects in subgroups KL 2, KL 3 and KL 2-3 and that this effect is maintained after 2 years of treatment. Interestingly, a surprisingly big effect is observed and maintained at timepoints ½, 1 and 2 years for KL grade 3 subjects. For KL 2 subjects with normal BMI the pain reducing effect it increases steadily from timepoints ½ to 1 and 2 years for KL grade 2 subjects. There is no effect seen with KL grade 4 subjects, but it should be noted that the number of subjects is very small.

Additionally, table 20B show that subjects experiencing at least 20 point reduction in pain is observed for normal weight subjects for KL 2, KL 3 and KL 2-3 subjects and that this effect is maintained after 2 years of treatment. The effect is even better for KL grade 2 subjects at 1 year and 2 years, compared to the level of KL grade 3. There is no effect seen with KL grade 4 subjects, but it should be noted that the number of subjects is very small.

### Results and Conclusion—Data on WOMAC Physical Function and Stiffness Pain—Normal BMI:

Surprisingly, results on pain parameters from WOMAC physical function and stiffness confirmed (data not shown) that normal BMI subjects subgroups KL 2, KL 3, KL 2-3 and KL 2-4 experienced a larger pain relief than the KL 4 normal BMI subjects subgroup at timepoints ½, 1 and 2 years. The only exception was KL grade 3 at 1 year for stiffness pain, wherein the pain relief was less than that of KL grade 4.

TABLE 20C
Subjects categorized with BMIs being overweight with at least 50%
reduction in transformed WOMAC pain subscale, or at least 12 or
20 points reduction in pain - subjects with KL grade 2, 3, 4, 2-3 and
2-4 - blinded phase - ITT analysis set

| | | | | | |
|---|---|---|---|---|---|
| | | | | | TOTAL |
| | KL | KL | KL | KL grade | (KL grade |
| | grade 2 | grade 3 | grade 4 | 2-3 | 2-4) |
| BMI overweight* | n/N (%) | n/N (%) | n/N (%) | n/N (%) | n/N (%) |

Week 26 (½ year)

| | | | | | |
|---|---|---|---|---|---|
| At least 50 % | 4/14 | 5/8 | 1/2 | 9/22 | 10/24 |
| reduction in pain | (28.6%) | (62.5%) | (50.0%) | (40.9%) | (41.7%) |
| At least 12 point | 19/31 | 11/21 | 3/9 | 30/52 | 33/61 |
| reduction in pain | (61.3%) | (52.4%) | (33.3%) | (57.7%) | (54.1%) |
| At least 20 point | 15/31 | 10/21 | 3/9 | 25/52 | 28/61 |
| reduction in pain | (48.4%) | (47.6%) | (33.3%) | (48.1%) | (45.9%) |

Week 52 (1 year)

| | | | | | |
|---|---|---|---|---|---|
| At least 50 % | 7/15 | 4/7 | 0/2 | 11/22 | 11/24 |
| reduction in pain | (46.7%) | (57.1%) | (00.0%) | (50.0%) | (45.8%) |
| At least 12 point | 17/30 | 7/19 | 3/8 | 24/49 | 27/57 |
| reduction in pain | (56.7%) | (36.8%) | (37.5%) | (49.0%) | (47.4%) |
| At least 20 point | 16/30 | 5/19 | 2/8 | 21/49 | 23/57 |
| reduction in pain | (53.3%) | (26.3%) | (25.0%) | (42.9%) | (40.4%) |

Week 104 (2 years)

| | | | | | |
|---|---|---|---|---|---|
| At least 50 % | 8/13 | 4/6 | 0/2 | 12/19 | 12/21 |
| reduction in pain | (61.5%) | (66.7%) | (00.0%) | (63.2%) | (57.1%) |
| At least 12 point | 13/23 | 8/17 | 3/5 | 21/40 | 24/45 |
| reduction in pain | (56.5%) | (47.1%) | (60.0%) | (52.5%) | (53.3%) |
| At least 20 point | 13/23 | 5/17 | 2/5 | 18/40 | 20/45 |
| reduction in pain | (56.5%) | (29.4%) | (40.0%) | (45.0%) | (44.4%) |

*Major adult BMI classifications are underweight (under 18.5 kg/m2), normal weight (18.5 to 24.9), overweight (25 to 29.9), and obese (30 or more).

### Conclusion Table 20C: Overweight BMI Subjects

Table 20C surprisingly show that at least 50% reduction in WOMAC pain is improved for overweight subjects for KL 2, KL 3 and KL 2-3 compared to the KL 4 subjects and that this effect is increasing over time i.e. after 2 years of treatment. It should be noted that the number of subjects for the KL4 group is rather small and may be used to consider trends.

Further, table 20C show that subjects experiencing at least 12 point reduction in pain is observed for overweight subjects for KL 2, KL 3 and KL 4 subjects and that this effect is maintained after 2 years of treatment. The effect is even better for KL grade 4 subjects at 2 years. Contrary, a surprising effect is observed with subjects experiencing at least 20 point reduction in pain, as the most subjects experiencing at least 20 point reduction in pain are KL grade 2 subjects compared to KL grade 4 patients at timepoints ½, 1 and 2 years. There is a lesser effect seen with KL grade 4 subjects, although 29% of the subjects experience at least 20 point reduction in pain after 2 years.

### Results and Conclusion-Data on WOMAC Physical Function and Stiffness Pain-BMI Overweight:

Surprisingly, results on pain parameters from WOMAC physical function confirmed (data not shown) that overweight BMI subjects subgroups KL 2, KL 3, KL 2-3 and KL 2-4 experienced a larger pain relief than the KL 4 overweight BMI subjects subgroup at timepoints 1 and 2 years.

Surprisingly, results on pain parameters from WOMAC stiffness confirmed (data not shown) that overweight BMI subjects subgroups KL 2, KL 3, KL 2-3 and KL 2-4 experienced a larger pain relief than the KL 4 overweight BMI subjects subgroup at timepoints 1 year. Additionally, KL 2 experienced a larger pain relief than KL 4 at timepoints ½ and 2 years. KL grade 2-3 and KL grade 2-4 also experienced a larger pain relief than KL 4 at ½ year.

TABLE 20D
Subjects categorized with BMIs being obese with at least 50%
reduction in transformed WOMAC pain subscale, or at least 12 or
20 points reduction in pain - subjects with KL grade 2, 3, 4, 2-3 and
2-4 - blinded phase - ITT analysis set

| | | | | | |
|---|---|---|---|---|---|
| | | | | | TOTAL |
| | KL | KL | KL | KL grade | (KL grade |
| | grade 2 | grade 3 | grade 4 | 2-3 | 2-4) |
| BMI obese* | n/N (%) | n/N (%) | n/N (%) | n/N (%) | n/N (%) |

Week 26 (½ year)

| | | | | | |
|---|---|---|---|---|---|
| At least 50 % | 7/18 | 4/9 | 2/2 | 11/27 | 13/29 |
| reduction in pain | (38.9%) | (44.4%) | 100.0%) | (40.7%) | (44.8%) |
| At least 12 point | 8/18 | 5/9 | 2/2 | 13/27 | 15/29 |
| reduction in pain | (44.4%) | (55.6%) | (100.0%) | (48.1%) | (51.7%) |
| At least 20 point | 8/18 | 5/9 | 2/2 | 13/27 | 15/29 |
| reduction in pain | (44.4%) | (55.6%) | (100.0%) | (48.1%) | (51.7%) |

Week 52 (1 year)

| | | | | | |
|---|---|---|---|---|---|
| At least 50 % | 5/16 | 5/9 | 0/1 | 10/25 | 10/26 |
| reduction in pain | (31.3%) | (55.6%) | (00.0%) | (40.0%) | (38.5%) |
| At least 12 point | 8/16 | 5/9 | 1/1 | 13/25 | 15/29 |
| reduction in pain | (50.0%) | (55.6%) | (100.0%) | (52.0%) | (51.7%) |
| At least 20 point | 4/16 | 5/9 | 1/1 | 9/25 | 10/26 |
| reduction in pain | (25.0%) | (55.6%) | (100.0%) | (36.0%) | (38.5%) |

Week 104 (2 years)

| | | | | | |
|---|---|---|---|---|---|
| At least 50 % | 4/11 | 5/9 | 1/1 | 9/20 | 10/21 |
| reduction in pain | (36.4%) | (55.6%) | (100.0%) | (45.0%) | (47.6%) |
| At least 12 point | 4/11 | 6/9 | 1/1 | 10/20 | 11/21 |
| reduction in pain | (36.4%) | (66.7%) | (100.0%) | (50.0%) | (52.4%) |
| At least 20 point | 3/11 | 5/9 | 1/1 | 8/20 | 9/21 |
| reduction in pain | (27.3%) | (55.6%) | (100.0%) | (40.0%) | (42.9%) |

*Major adult BMI classifications are underweight (under 18.5 kg/m2), normal weight (18.5 to 24.9), overweight (25 to 29.9), and obese (30 or more).

### Conclusion Table 20D: Obese BMI Subjects

Table 20D surprisingly show that at least 50% reduction in WOMAC pain is observed for obese subjects for KL 2, KL 3 and KL 4 subgroups at 1 and 2 years and that this effect is maintained after 2 years of treatment. The effect is even better for KL grade 3 subjects, that maintain its high level of about 55% obtained after 1 year and 2 years. The number of subjects for the KL4 group is to small to consider.

Further, table 20D surprisingly show that subjects experiencing at least 12 point reduction in pain is observed for obese subjects for KL 2, KL 3 and KL 4 subjects and that this effect is maintained after 2 years of treatment. The effect is even better for KL grade 3 subjects at 2 years. The number of subjects for the KL4 group is to small to consider. Similar surprising effects are observed with subjects experiencing at least 20 point reduction in pain where the effect is even better for KL grade 3 subjects is maintained equally high at around 55% at timepoints ½, 1 and 2 years.

### Results and Conclusion-Data on WOMAC Physical Function and Stiffness Pain—BMI Obese:

Surprisingly, results on pain parameters from WOMAC physical function and stiffness confirmed (data not shown) that obese BMI subjects from subgroups KL 2, KL 3, KL 2-3 and KL 2-4 experienced a large pain relief at timepoints ½, 1 and 2 years. The number of subjects for the KL4 group was to small to consider (N=1).

### Age

### Aim of Study:

Aim of study was to investigate the WOMAC pain effect in specific age subgroups of KL grade 2, 3, 4, 2-3 and 2-4 subgroup subjects.

TABLE 21
Summary of transformed WOMAC pain subscale (0-100) by age group -
age < 50 years - blinded phase - ITT analysis set - total KL grades 2-4.

| | age < 50 years | |
|---|---|---|
| | | Change from |
| | Actual values | baseline |
| ITT analysis set (N,%) | 8 (100.0) | |
| Baseline | | |
| N | 8 | |
| Mean (SD) | 46.9 (13.9) | |
| Median | 47.5 | |
| Min-Max | 25-70 | |
| Week 4 | | |
| N | 8 | 8 |
| Mean (SD) | 35.6 (27.0) | −11.3 (36.4) |
| Median | 30.0 | −22.5 |
| Min-Max | 10-100 | −40-75 |
| Week 12 | | |
| N | 8 | 8 |
| Mean (SD) | 28.8 (15.5) | −18.1 (21.5) |
| Median | 25.0 | −17.5 |
| Min-Max | 15-65 | −50-20 |
| Week 26 | | |
| N | 7 | 7 |
| Mean (SD) | 22.1 (16.3) | −27.9 (19.3) |
| Median | 20.0 | −35.0 |
| Min-Max | 0-50 | −50-5 |
| Week 52 | | |
| N | 7 | 7 |
| Mean (SD) | 13.6 (9.0) | −36.4 (17.3) |
| Median | 10.0 | −40.0 |
| Min-Max | 0-25 | −60-−15 |

N: Number of subjects,
SD: Standard deviation

TABLE 22
Summary of transformed WOMAC pain subscale (0-100) by age group -
age 50-59 years - blinded phase - ITT analysis set - total KL grades 2-4.

| | age 50-59 years | |
|---|---|---|
| | | Change from |
| | Actual values | baseline |
| ITT analysis set (N,%) | 15 (100.0) | |
| Baseline | | |
| N | 15 | |
| Mean (SD) | 48.0 (14.6) | |
| Median | 45.0 | |
| Min-Max | 25-70 | |
| Week 4 | | |
| N | 15 | 15 |
| Mean (SD) | 38.7 (16.5) | −9.3 (16.4) |
| Median | 35.0 | −10.0 |
| Min-Max | 25-70 | −35-35 |
| Week 12 | | |
| N | 14 | 14 |
| Mean (SD) | 33.9 (13.2) | −15.7 (22.2) |
| Median | 35.0 | −15.0 |
| Min-Max | 5-55 | −60-25 |
| Week 26 | | |
| N | 14 | 14 |
| Mean (SD) | 33.9 (20.9) | −15.7 (18.2) |
| Median | 27.5 | −15.0 |
| Min-Max | 10-75 | −50-15 |
| Week 52 | | |
| N | 10 | 10 |
| Mean (SD) | 25.0 (14.1) | −19.5 (18.9) |
| Median | 22.5 | −15.0 |
| Min-Max | 5-55 | −55-5 |

N: Number of subjects,
SD: Standard deviation

TABLE 23
Summary of transformed WOMAC pain subscale (0-100) by age group -
age 60-69 years - blinded phase - ITT analysis set - total KL grades 2-4.

| | age 60-69 years | |
|---|---|---|
| | | Change from |
| | Actual values | baseline |
| ITT analysis set | 47 (100.0) | |
| (N,%) | | |
| Baseline | | |
| N | 47 | |
| Mean (SD) | 43.5 (11.8) | |
| Median | 45.0 | |
| Min-Max | 20-70 | |
| Week 4 | | |
| N | 47 | 47 |
| Mean (SD) | 30.1 (16.8) | −13.4 (15.8) |
| Median | 30.0 | −10.0 |
| Min-Max | 0-65 | −55-25 |
| Week 12 | | |
| N | 47 | 47 |
| Mean (SD) | 22.4 (16.5) | −21.1 (18.6) |
| Median | 25.0 | −20.0 |
| Min-Max | 0-60 | −65-25 |
| Week 26 | | |
| N | 47 | 47 |
| Mean (SD) | 23.9 (15.2) | −19.6 (17.2) |
| Median | 25.0 | −20.0 |
| Min-Max | 0-55 | −65-20 |
| Week 52 | | |
| N | 46 | 46 |
| Mean (SD) | 23.6 (17.9) | −19.9 (19.6) |
| Median | 20.0 | −15.0 |
| Min-Max | 0-65 | −65-10 |

N: Number of subjects,
SD: Standard deviation

TABLE 24
Summary of transformed WOMAC pain subscale (0-100) by age group -
age ≥ 70 years - blinded phase - ITT analysis set - total KL grades 2-4.

| | age ≥ 70 years | |
|---|---|---|
| | | Change from |
| | Actual values | baseline |
| ITT analysis set (N,%) | 49 (100.0) | |
| Baseline | | |
| N | 49 | |
| Mean (SD) | 45.4 (14.6) | |
| Median | 45.0 | |
| Min-Max | 10-75 | |
| Week 4 | | |
| N | 49 | 49 |
| Mean (SD) | 32.7 (18.1) | −12.8 (18.8) |
| Median | 30.0 | −10.0 |
| Min-Max | 0-70 | −65-20 |
| Week 12 | | |
| N | 46 | 46 |
| Mean (SD) | 26.4 (17.5) | −19.1 (18.8) |
| Median | 25.0 | −20.0 |
| Min-Max | 0-60 | −55-15 |
| Week 26 | | |
| N | 46 | 46 |
| Mean (SD) | 28.2 (19.4) | −16.6 (20.9) |
| Median | 25.0 | −10.0 |
| Min-Max | 0-75 | −65-20 |
| Week 52 | | |
| N | 44 | 44 |
| Mean (SD) | 31.4 (22.1) | −13.2 (23.0) |
| Median | 30.0 | −10.0 |
| Min-Max | 0-75 | −65-20 |

N: Number of subjects,
SD: Standard deviation

TABLE 25
Analysis of change from baseline to week 52 in transformed
WOMAC pain subscale (0-100) by age group - blinded
phase - ITT analysis set - total KL grades 2-4.

| | N | LSMean (95% CI) | |
|---|---|---|---|
| Age <50 years | | | |
| Week 4 | | | |
| PAAG-OA | 8 | −12.75 | (−28.38; 2.88) |
| Week 13 | | | |
| PAAG-OA | 8 | −18.39 | (−28.00; −8.79) |
| Week 26 | | | |
| PAAG-OA | 7 | −18.97 | (−36.51; −1.44) |
| Week 52 | | | |
| PAAG-OA | 7 | −31.02 | (−45.40; −16.63) |

N: Number of subjects contributing to the analysis
The analysis is performed on change from baseline using a mixed model for repeated measures including fixed, categorical effects of treatment, week, treatment-by-week interaction and site, as well as the baseline value and baseline-by-week interaction as covariates.

TABLE 26
Analysis of change from baseline to week 52 in transformed
WOMAC pain subscale (0-100) by age group - blinded
phase - ITT analysis set - total KL grades 2-4.

| | N | LSMean (95% CI) | |
|---|---|---|---|
| Age 50-60 years | | | |
| Week 4 | | | |
| PAAG-OA | 15 | −10.60 | (−18.21; −3.00) |
| Week 13 | | | |
| PAAG-OA | 14 | −14.11 | (−22.15; −6.07) |
| Week 26 | | | |
| PAAG-OA | 14 | −14.76 | (−24.90; −4.61) |
| Week 52 | | | |
| PAAG-OA | 10 | −22.01 | (−32.42; −11.60) |

N: Number of subjects contributing to the analysis
The analysis is performed on change from baseline using a mixed model for repeated measures including fixed, categorical effects of treatment, week, treatment-by-week interaction and site, as well as the baseline value and baseline-by-week interaction as covariates.

TABLE 27
Analysis of change from baseline to week 52 in transformed
WOMAC pain subscale (0-100) by age group - blinded
phase - ITT analysis set - total KL grades 2-4.

| | N | LSMean (95% CI) | |
|---|---|---|---|
| Age 60-70 years | | | |
| Week 4 | | | |
| PAAG-OA | 47 | −14.03 | (−18.49; −9.58) |
| Week 13 | | | |
| PAAG-OA | 47 | −22.15 | (−26.70; −17.60) |
| Week 26 | | | |
| PAAG-OA | 47 | −20.57 | (−25.44; −15.70) |
| Week 52 | | | |
| PAAG-OA | 46 | −20.67 | (−25.84; −15.50) |

N: Number of subjects contributing to the analysis
The analysis is performed on change from baseline using a mixed model for repeated measures including fixed, categorical effects of treatment, week, treatment-by-week interaction and site, as well as the baseline value and baseline-by-week interaction as covariates.

TABLE 28
Analysis of change from baseline to week 52 in transformed
WOMAC pain subscale (0-100) by age group - blinded
phase - ITT analysis set - total KL grades 2-4.

| | N | LSMean (95% CI) | |
|---|---|---|---|
| Age ≥70 years | | | |
| Week 4 | | | |
| PAAG-OA | 49 | −12.64 | (−17.31; −7.98) |
| Week 13 | | | |
| PAAG-OA | 46 | −18.57 | (−23.10; −14.04) |
| Week 26 | | | |
| PAAG-OA | 46 | −16.71 | (−21.67; −11.75) |
| Week 52 | | | |
| PAAG-OA | 44 | −13.42 | (−18.65; −8.19) |

N: Number of subjects contributing to the analysis
The analysis is performed on change from baseline using a mixed model for repeated measures including fixed, categorical effects of treatment, week, treatment-by-week interaction and site, as well as the baseline value and baseline-by-week interaction as covariates.

TABLE 29
Analysis of change from baseline to week 52 in transformed WOMAC
pain subscale by age group - ITT - total KL grades 2-4.

| | N | LSMeans (95% CI) | |
|---|---|---|---|
| Age <50 years | | | |
| PAAG-OA | 7 | −31.02 | (−45.40; −16.63) |
| Age 50-59 years | | | |
| PAAG-OA | 10 | −22.01 | (−32.42; −11.60) |
| Age 60-69 years | | | |
| PAAG-OA | 46 | −20.67 | (−25.84; −15.50) |
| Age 70-79 years | | | |
| PAAG-OA | 34 | −12.76 | (−18.67; −6.84) |
| Age ≥80 years | | | |
| PAAG-OA | 10 | −15.78 | (−29.45; −2.11) |

TABLE 30
Analysis of change from baseline to week 52 in transformed WOMAC
pain subscale by age group - ITT - total KL grades 2-4.

| | N | LSMeans (95% CI) | |
|---|---|---|---|
| Age <50 years | | | |
| PAAG-OA | 7 | −31.02 | (−45.40; −16.63) |
| Age 50-59 years | | | |
| PAAG-OA | 10 | −22.01 | (−32.42; −11.60) |
| Age 60-69 years | | | |
| PAAG-OA | 46 | −20.67 | (−25.84; −15.50) |
| Age ≥70 years | | | |
| PAAG-OA | 44 | −13.42 | (−18.65; −8.19) |

N: Number of subjects contributing to the analysis
The analysis is performed on change from baseline using a mixed model for repeated measures including fixed, categorical effects of treatment, week, treatment-by-week interaction and site, as well as the baseline value and baseline-by-week interaction as covariates.

TABLE 31
Analysis of change from baseline to week 52 in transformed WOMAC
pain subscale by age group - ITT - total KL grades 2-4.

| | N | LSMeans (95% CI) | |
|---|---|---|---|
| Age <70 years | | | |
| PAAG-OA | 63 | −21.25 | (−25.52; −16.97) |
| Age ≥70 years | | | |
| PAAG-OA | 44 | −13.42 | (−18.65; −8.19) |

N: Number of subjects contributing to the analysis
The analysis is performed on change from baseline using a mixed model for repeated measures including fixed, categorical effects of treatment, week, treatment-by-week interaction and site, as well as the baseline value and baseline-by-week interaction as covariates.

### Conclusion

Tables 21-31 demonstrate that age influences the ability of the total KL grades 2-4 subjects to benefit from the treatment with PAAG-OA.

To further analyze the age effect on the subgroups of KL grades, the following data is presented in tables 32A-32B:

TABLE 32A
Subjects less than 70 years old with at least 50% reduction
in transformed WOMAC pain subscale, or at least 12 or 20
points reduction in pain - subjects with KL grade 2, 3,
4, 2-3 and 2-4 - blinded phase - ITT analysis set

| | | | | | |
|---|---|---|---|---|---|
| | | | | | TOTAL |
| | KL | KL | KL | KL | (KL |
| Age (less than | grade 2 | grade 3 | grade 4 | grade 2-3 | grade 2-4) |
| 70 years old) | n/N (%) | n/N (%) | n/N (%) | n/N (%) | n/N (%) |

Week 26 (½ year)

| | | | | | |
|---|---|---|---|---|---|
| At least 50% | 18/40 | 8/25 | 3/3 | 26/65 | 29/68 |
| reduction in pain | (45.0%) | (32.0%) | (100.0%) | (40.0%) | (42.6%) |
| At least 12 point | 25/40 | 15/25 | 2/3 | 40/65 | 42/68 |
| reduction in pain | (62.5%) | (60.0%) | (66.7%) | (61.5%) | (61.8%) |
| At least 20 point | 21/40 | 14/25 | 2/3 | 35/65 | 37/68 |
| reduction in pain | (52.5%) | (56.0%) | (66.7%) | (53.8%) | (54.4%) |

Week 52 (1 year)

| | | | | | |
|---|---|---|---|---|---|
| At least 50% | 19/37 | 8/23 | 2/3 | 27/60 | 29/63 |
| reduction in pain | (51.4%) | (34.8%) | (66.7%) | (45.0%) | (46.0%) |
| At least 12 point | 27/37 | 11/23 | 2/3 | 38/60 | 40/63 |
| reduction in pain | (73.0%) | (47.8%) | (66.7%) | (63.3%) | (63.5%) |
| At least 20 point | 22/37 | 7/23 | 1/3 | 29/60 | 30/63 |
| reduction in pain | (59.5%) | (30.4%) | (33.3%) | (48.3%) | (47.6%) |

Week 104 (2 years)

| | | | | | |
|---|---|---|---|---|---|
| At least 50% | 18/31 | 10/21 | 1/3 | 28/52 | 29/55 |
| reduction in pain | (58.1%) | (47.6%) | (33.3%) | (53.8%) | (52.7%) |
| At least 12 point | 21/31 | 13/21 | 2/3 | 34/52 | 36/55 |
| reduction in pain | (67.7%) | (61.9%) | (66.7%) | (65.4%) | (65.5%) |
| At least 20 point | 20/31 | 9/21 | 2/3 | 29/52 | 31/55 |
| reduction in pain | (64.5%) | (42.9%) | (66.7%) | (55.8%) | (56.4%) |

Conclusion—Age Less than 70 Years Old Subjects

Table 32A surprisingly show that at least 50% reduction in WOMAC pain is observed for subjects less than 70 years old for KL 2 and/or KL 3 subjects after 2 years of treatment. The most pronounced effect is seen for KL grade 2 subjects being less than 70 years old compared to KL 4 subjects being less than 70 years old.

Further, table 32A show that at least 60% of subjects being less than 70 years old experiencing at least 12 point reduction in pain, which is seen for all subgroups at ½ year of treatment. These levels are maintained after 2 years of treatment. The percentage of subjects experiencing at least 12 point pain reducing effect is even better for KL grade 2 subjects at 2 years. A minor improvement for KL grade 3 is also observed at 2 years, compared to ½ year, whereas the initial level for KL 4 subjects is maintained after 2 years.

A large percentage of subjects less than 70 years old experiencing at least 20 point reduction in pain is shown for KL 2, 3 and 4 patients at ½ years after treatment. The percentage of subjects experiencing at least 20 point reduction in pain is further improved for KL 2 subjects at 2 years (64.5%), whereas the level for KL 3 subjects have decreased to ca. 43%). The KL 4 level is unchanged at 2 years compared to ½ year.

Results and Conclusion—Data on WOMAC Physical Function—Age Less than 70 Years Old:

Surprisingly, results on pain parameters from WOMAC physical function confirmed (data not shown) that subjects being less than 70 years old subgroups KL 2 experienced a larger pain relief than the KL 4 subjects being less than 70 years old subgroup at timepoints 1 and 2 years.

TABLE 32B
Subjects more than 70 years old with at least 50% reduction
in transformed WOMAC pain subscale, or at least 12 or 20
points reduction in pain - subjects with KL grade 2, 3,
4, 2-3 and 2-4 - blinded phase - ITT analysis set

| | | | | | |
|---|---|---|---|---|---|
| | | | | | TOTAL |
| | KL | KL | KL | KL | (KL |
| Age (more than | grade 2 | grade 3 | grade 4 | grade 2-3 | grade 2-4) |
| 70 years old) | n/N (%) | n/N (%) | n/N (%) | n/N (%) | n/N (%) |

Week 26 (½ year)

| | | | | | |
|---|---|---|---|---|---|
| At least 50% | 9/23 | 7/13 | 3/10 | 16/36 | 19/46 |
| reduction in pain | (39.1%) | (53.8%) | (30.0%) | (44.4%) | (41.3%) |
| At least 12 point | 10/23 | 8/13 | 3/10 | 18/36 | 21/46 |
| reduction in pain | (43.5%) | (61.5%) | (30.0%) | (50.0%) | (45.7%) |
| At least 20 point | 8/23 | 8/13 | 3/10 | 16/36 | 19/46 |
| reduction in pain | (34.8%) | (61.5%) | (30.0%) | (44.4%) | (41.3%) |

Week 52 (1 year)

| | | | | | |
|---|---|---|---|---|---|
| At least 50% | 8/24 | 6/12 | 1/8 | 14/36 | 15/44 |
| reduction in pain | (33.3%) | (50.0%) | (12.5%) | (38.9%) | (34.1%) |
| At least 12 point | 9/24 | 7/12 | 2/8 | 16/36 | 18/44 |
| reduction in pain | (37.5%) | (58.3%) | (25.0%) | (44.4%) | (40.9%) |
| At least 20 point | 7/24 | 6/12 | 2/8 | 13/36 | 15/44 |
| reduction in pain | (29.2%) | (50.0%) | (25.0%) | (36.1%) | (34.1%) |

Week 104 (2 years)

| | | | | | |
|---|---|---|---|---|---|
| At least 50% | 8/16 | 4/11 | 1/5 | 12/27 | 13/32 |
| reduction in pain | (50.0%) | (36.4%) | (20.0%) | (44.4%) | (40.6%) |
| At least 12 point | 7/16 | 6/11 | 2/5 | 13/27 | 15/32 |
| reduction in pain | (43.8%) | (54.5%) | (40.0%) | (48.1%) | (46.9%) |
| At least 20 point | 6/16 | 5/11 | 1/5 | 11/27 | 31/55 |
| reduction in pain | (37.5%) | (45.5%) | (20.0%) | (40.7%) | (56.4%) |

Conclusion—Age More than 70 Years Old Subjects

Table 32B surprisingly show that at least 50% reduction in WOMAC pain is observed for subjects more than 70 years old for KL 2, KL 3, KL 2-3 and KL 2-4 subjects after ½, 1 and 2 years of treatment compared to KL 4 subjects more than 70 years old. The most pronounced effect is seen for KL grade 3 subjects being more than 70 years old compared to KL 4 subjects being more than 70 years old.

The same results are consistently seen for KL 2, KL 3, KL 2-3 and KL 2-4 subjects after ½, 1 and 2 years of treatment compared to KL 4 subjects more than 70 years old with regard to the subjects having at least 12 or 20 point reduction in pain. Again, the most pronounced effect is seen with KL 3 subjects compared to KL 4 subjects.

Results and Conclusion—Data on WOMAC Physical Function and Stiffness—Age More than 70 Years Old:

Surprisingly, results on pain parameters from WOMAC physical function confirmed (data not shown) that subjects being more than 70 years old subgroups KL 3, KL 2-3 and KL 2-4 experienced a larger pain relief than the KL 4 subjects being more than 70 years old subgroup at timepoints ½, 1 and 2 years. The KL2 subgroup experienced a larger pain relief than the KL 4 subjects being more than 70 years old subgroup at timepoints 1 and 2 years.

Surprisingly, results on pain parameters from WOMAC stiffness confirmed (data not shown) that subjects being more than 70 years old subgroups KL 2, KL 3, KL 2-3 and KL 2-4 experienced a larger pain relief than the KL 4 subjects being more than 70 years old subgroup at timepoints ½, 1 and 2 years.

Comparison with Hyalonic Acid

TABLE 33
Post Hoc: Analysis of change from baseline to week 26 in transformed
WOMAC pain subscale in selected subgroups - ITT - KL grade 2 or 3.

| | | | |
|---|---|---|---|
| | LSMeans | Treatment difference | |
| N | (95% CI) | (95% CI) | p-value |

KL grade 2 or 3

| | | | | | | |
|---|---|---|---|---|---|---|
| Synvisc-One | 99 | −15.65 | (−19.14; −12.17) | | | |
| PAAG-OA | 101 | −18.82 | (−22.24; −15.39) | 3.16 | (−1.73; 8.05) | 0.2041 |

Age <70 years

| | | | | | | |
|---|---|---|---|---|---|---|
| Synvisc-One | 67 | −15.74 | (−19.89; −11.58) | | | |
| PAAG-OA | 68 | −19.66 | (−23.78; −15.55) | 3.93 | (−1.93; 9.79) | 0.1872 |

KL grade 2 or 3 and age <70 years

| | | | | | | |
|---|---|---|---|---|---|---|
| Synvisc-One | 58 | −16.58 | (−21.11; −12.06) | | | |
| PAAG-OA | 65 | −19.44 | (−23.70; −15.17) | 2.85 | (−3.40; 9.11) | 0.3679 |

N: Number of subjects contributing to the analysis
The analysis is performed on change from baseline using a mixed model for repeated measures including fixed, categorical effects of treatment, week, treatment-by-week interaction and site, as well as the baseline value and baseline-by-week interaction as covariates.

TABLE 34
Post Hoc: Analysis of change from baseline to week 52 in transformed
WOMAC pain subscale in selected subgroups - ITT - KL grade 2 or 3.

| | | | |
|---|---|---|---|
| | LSMeans | Treatment difference | |
| N | (95% CI) | (95% CI) | p-value |

KL grade 2 or 3

| | | | | | | |
|---|---|---|---|---|---|---|
| Synvisc-One | 94 | −13.37 | (−17.01; −9.73) | | | |
| PAAG-OA | 96 | −18.94 | (−22.52; −15.35) | 5.57 | (0.45; 10.69) | 0.0332 |

Age <70 years

| | | | | | | |
|---|---|---|---|---|---|---|
| Synvisc-One | 62 | −13.95 | (−18.26; −9.64) | | | |
| PAAG-OA | 63 | −21.25 | (−25.52; −16.97) | 7.30 | (1.20; 13.40) | 0.0195 |

KL grade 2 or 3 and age <70 years

| | | | | | | |
|---|---|---|---|---|---|---|
| Synvisc-One | 54 | −13.77 | (−18.48; −9.06) | | | |
| PAAG-OA | 60 | −21.49 | (−25.95; −17.04) | 7.73 | (1.18; 14.27) | 0.0212 |

N: Number of subjects contributing to the analysis. The analysis is performed on change from baseline using a mixed model for repeated measures including fixed, categorical effects of treatment, week, treatment-by-week interaction and site, as well as the baseline value and baseline-by-week interaction as covariates.

### Conclusion

Tables 33 and 34 demonstrate a comparison of—KL grade 2 or 3 subjects treated with either PAAG-OA or Synvisc-One, which is a hyalonic acid gel, and their experience of pain 26 weeks and 52 weeks after treatment. It is shown that looking at subjects diagnosed with KL grade 2 or 3 and/or subjects age<70 years, both PAAG-OA or Synvisc-One were able to reduce the pain. However, PAAG-OA showed a better pain reduction after 52 weeks.

Results with Several Combinations of Subgroups of Subjects

In the following, selected data is presented wherein two factors are combined with KL grades.

TABLE 35
Subjects less than 70 years old with a BMI categorized
“overweight” having at least 50% reduction in
transformed WOMAC pain subscale - subjects with KL grade
2, 3, 4, 2-3 and 2-4 - blinded phase - ITT analysis set

| | | | | | |
|---|---|---|---|---|---|
| Age (less than | | | | | TOTAL |
| 70 years old) | KL | KL | KL | KL | (KL |
| and BMI | grade 2 | grade 3 | grade 4 | grade 2-3 | grade 2-4) |
| “overweight” | n/N (%) | n/N (%) | n/N (%) | n/N (%) | n/N (%) |

Week 26 (½ year)

| | | | | | |
|---|---|---|---|---|---|
| At least 50% | 12/17 | 1/14 | 2/2 | 13/31 | 15/33 |
| reduction in pain | (70.6%) | (7.1%) | (100.0%) | (41.9%) | (45.5%) |

Week 52 (1 year)

| | | | | | |
|---|---|---|---|---|---|
| At least 50% | 12/16 | 2/13 | 2/2 | 14/29 | 16/31 |
| reduction in pain | (75.0%) | (15.4%) | (100.0%) | (48.3%) | (51.6%) |

Week 104 (2 years)

| | | | | | |
|---|---|---|---|---|---|
| At least 50% | 10/14 | 3/12 | 1/2 | 13/26 | 14/28 |
| reduction in pain | (71.4%) | (25.0%) | (50.0%) | (50.0%) | (50.0%) |

BMI Overweight: 25-29.9 kg/m²

Conclusion—Overweight Subjects Less than 70 Years Old

It is clear from the data in table 35 that at the timepoint of 2 years a higher percentage of KL grade 2 subjects being overweight and less than 70 years old experience at least 50% reduction in pain compared to KL grade 4 and 3 subjects. Remarkably, KL grade 2 subjects had a surprisingly much larger pain reduction than KL grade 3 subjects at timepoints ½, 1 and 2 years. KI grade 2-3 patients had a similar pain effect at 2 years compared to KL grade 4.

TABLE 36
Subjects more than 70 years old with a BMI categorized
“overweight” having at least 50% reduction in
transformed WOMAC pain subscale - subjects with KL grade
2, 3, 4, 2-3 and 2-4 - blinded phase - ITT analysis set

| | | | | | |
|---|---|---|---|---|---|
| | | | | | TOTAL |
| Age (more than 70 | KL | KL | KL | KL | (KL |
| years old) and BMI | grade 2 | grade 3 | grade 4 | grade 2-3 | grade 2-4) |
| “overweight” | n/N (%) | n/N (%) | n/N (%) | n/N (%) | n/N (%) |

Week 26 (½ year)

| | | | | | |
|---|---|---|---|---|---|
| At least 50% | 4/14 | 5/7 | 1/7 | 9/21 | 10/28 |
| reduction in pain | (28.6%) | (71.4%) | (14.3%) | (42.9%) | (35.7%) |

Week 52 (1 year)

| | | | | | |
|---|---|---|---|---|---|
| At least 50% | 3/14 | 3/6 | 1/6 | 6/20 | 7/26 |
| reduction in pain | (21.4%) | (50.0%) | (16.7%) | (30.0%) | (26.9%) |

Week 104 (2 years)

| | | | | | |
|---|---|---|---|---|---|
| At least 50% | 4/9 | 2/5 | 0/3 | 6/14 | 6/17 |
| reduction in pain | (44.4%) | (40.0%) | (00.0%) | (42.9%) | (35.3%) |

BMI Overweight: 25-29.9 kg/m²

Conclusion—Overweight Subjects More than 70 Years Old

It is surprisingly shown from the data in table 36 that at the timepoint of ½, 1 and 2 years a higher percentage of KL grade 2, 3 and 2-3 subjects being overweight and more than 70 years old experience at least 50% reduction in pain compared to KL grade 2 subjects.

Similar results were obtained with subjects categorized with being more than 70 years old with a BMI categorized “overweight” having at least 12 or 20 points reduction in pain (data not shown here).

Results and Conclusion—Data on WOMAC Physical Function and Stiffness-Overweight Subjects More than 70 Years Old:

Surprisingly, results on pain parameters from WOMAC physical function confirmed (data not shown) that subjects being overweight and more than 70 years old of subgroups KL 3, KL 2-3 and KL 2-4 experienced a larger pain relief than the KL 4 subjects being overweight and more than 70 years old subgroup at timepoints ½, 1 and 2 years. The KL 2 subjects being overweight and more than 70 years old subgroup was also superior to the KL 4 subjects being overweight and more than 70 years old subgroup at timepoint 2 years.

Surprisingly, results on pain parameters from WOMAC stiffness confirmed (data not shown) that the subjects being overweight and more than 70 years old subgroups KL 2, KL3, KL 2-3 and KL 2-4 experienced a larger pain relief than the KL 4 subjects being overweight and more than 70 years old subgroup at timepoints ½ and 1 year. The KL 3 subjects being overweight and more than 70 years old subgroup was also superior to the KL 4 subjects being overweight and more than 70 years old subjects at timepoint 2 years.

TABLE 37
Male subjects more than 70 years old having at least 50% reduction
in transformed WOMAC pain subscale - subjects with KL grade
2, 3, 4, 2-3 and 2-4 - blinded phase - ITT analysis set.

| | | | | | |
|---|---|---|---|---|---|
| | | | | | TOTAL |
| Age (more than | KL | KL | KL | KL | (KL |
| 70 years old) and | grade 2 | grade 3 | grade 4 | grade 2-3 | grade 2-4) |
| male | n/N (%) | n/N (%) | n/N (%) | n/N (%) | n/N (%) |

Week 26 (½ year)

| | | | | | |
|---|---|---|---|---|---|
| At least 50% | 3/11 | 1/3 | 2/8 | 4/14 | 6/22 |
| reduction in pain | (27.3%) | (33.3%) | (25.0%) | (28.6%) | (27.3%) |

Week 52 (1 year)

| | | | | | |
|---|---|---|---|---|---|
| At least 50% | 5/11 | 1/2 | 1/7 | 6/13 | 7/20 |
| reduction in pain | (45.5%) | (50.0%) | (14.3%) | (46.2%) | (35.0%) |

Week 104 (2 years)

| | | | | | |
|---|---|---|---|---|---|
| At least 50% | 5/7 | 1/2 | 1/4 | 6/9 | 7/13 |
| reduction in pain | (71.4%) | (50.0%) | (25.0%) | (66.7%) | (53.8%) |

Conclusion—Male Subjects More than 70 Years Old

It is clear from the data in table 37 that at the timepoint of ½, 1 and 2 years a surprisingly higher percentage of KL grade 2, 3 and/or 2-3 male subjects more than 70 years old experience at least 50% reduction in pain compared to the percentage experienced for KL grade 4 male subjects more than 70 years old.

Similar surprising results were obtained with male subjects more than 70 years old having at least 12 or 20 points reduction in pain (data not shown here) i.e. at the timepoint of ½, 1 and 2 years a surprisingly higher percentage of KL grade 2 and/or 2-3 male subjects more than 70 years old experience at least 12 or 20 points reduction in pain compared to the percentage experienced for KL grade 4 male subjects more than 70 years old. KL 3 subjects also followed this pattern with the exception of timepoint 2 years for at least 12 points reduction in pain, as the percentage of pain reduction subjects were identical to KL grade 4 (i.e. 50%). However, the number of subjects in the KL 3 group was only 2 subjects at 2 years, which may explain this observation.

Conclusion—Data on WOMAC Physical Function and Stiffness-Male Subjects More than 70 Years Old:

Surprisingly, results on pain parameters from WOMAC physical function confirmed (data not shown) that male subjects more than 70 years old of subgroups KL 2, KL3, KL 2-3 and KL 2-4 experienced a larger pain relief than the KL 4 male subjects more than 70 years old subgroup at timepoints ½, 1 and 2 years.

Surprisingly, results on pain parameters from WOMAC stiffness confirmed (data not shown) that male subjects more than 70 years old of subgroups KL 2, KL 2-3 and KL 2-4 experienced a larger pain relief than the KL 4 male subjects more than 70 years old subgroup at timepoints ½, 1 and 2 years. The KL 3 male subjects more than 70 years old subgroup was also superior to KL 4 male subjects more than 70 years old subgroup at timepoint ½ and 1 year.

TABLE 38
Male subjects with a BMI categorized “overweight” having
at least 50% reduction in transformed WOMAC pain subscale - subjects
with KL grade 2, 3, 4, 2-3 and 2-4 - blinded phase - ITT analysis set

| | | | | | |
|---|---|---|---|---|---|
| | | | | | TOTAL |
| | KL | KL | KL | KL | (KL |
| Males and BMI | grade 2 | grade 3 | grade 4 | grade 2-3 | grade 2-4) |
| “overweight” | n/N (%) | n/N (%) | n/N (%) | n/N (%) | n/N (%) |

Week 26 (½ year)

| | | | | | |
|---|---|---|---|---|---|
| At least 50% | 8/15 | 1/10 | 2/8 | 9/25 | 11/33 |
| reduction in pain | (53.3%) | (10.0%) | (25.0%) | (36.0%) | (33.3%) |

Week 52 (1 year)

| | | | | | |
|---|---|---|---|---|---|
| At least 50% | 8/15 | 2/9 | 3/8 | 10/24 | 13/32 |
| reduction in pain | (53.3%) | (22.2%) | (37.5%) | (41.7%) | (40.6%) |

Week 104 (2 years)

| | | | | | |
|---|---|---|---|---|---|
| At least 50% | 7/11 | 3/8 | 1/5 | 10/19 | 11/24 |
| reduction in pain | (63.6%) | (37.5%) | (20.0%) | (52.6%) | (45.8%) |

BMI Overweight: 25-29.9 kg/m²

### Conclusion—Overweight Male Subjects

It is shown from the data in table 38 that at the timepoint of ½, 1 and 2 years a higher percentage of KL grade 2 and KL grade 2-3 overweight male subjects experience at least 50% reduction in pain compared to KL grade 4 subjects. A higher percentage of overweight male KL 3 subjects experience at least 50% reduction in pain compared to KL grade 4 subjects at 2 years.

Similar results were obtained with overweight male subjects having at least 12 or 20 points reduction in pain (data not shown here) for KL grade 2 subjects compared to KL 4 subjects at the timepoint of ½, 1 and 2 years. For KL grade 2-3 subjects, similar effects were observed with overweight male subjects experiencing at least 12 points reduction in pain but only for timepoints ½ year and 1 year, whereas a higher percentage of overweight male subjects experienced at least 20 points reduction in pain at timepoint of ½, 1 and 2 years.

### Conclusion—Data on WOMAC Physical Function and Stiffness—Overweight Males:

Surprisingly, results on pain parameters from WOMAC physical function confirmed (data not shown) that male subjects being overweight and belonging to subgroups KL 2, KL 2-3 and KL 2-4 experienced a larger pain relief than the KL 4 overweight male subjects subgroup at timepoints ½, 1 and 2 years. The KL 3 subgroup was also superior to KL 4 overweight male subjects subgroup at timepoints 1 and 2 years.

Surprisingly, results on pain parameters from WOMAC stiffness confirmed (data not shown) demonstrate that overweight male subjects belonging to subgroups KL 2, KL 2-3 and KL 2-4 experienced a larger pain relief than the KL 4 overweight male subjects at timepoints ½, 1 and 2 years. The KL 3 overweight male subjects subgroup was also superior to the KL 4 overweight male subjects at timepoint 1 year.

### REFERENCES

Kellgreen J H, Lawrence J S (1957) Radiological assessment of ostoearthrosis. Ann Rheum Dis 16:494-501.

1-34. (canceled)

35. A polyacrylamide hydrogel for use in prevention and/or treatment of osteoarthritis for at least 104 weeks in a human diagnosed with KL grade 2 and/or KL grade 3 osteoarthritis.

36. The polyacrylamide hydrogel for use according to claim 35, wherein said human is diagnosed with KL grade 2 osteoarthritis.

37. The polyacrylamide hydrogel for use according to claim 35, wherein said human is diagnosed with KL grade 3 osteoarthritis.

38. The polyacrylamide hydrogel for use according to claim 35, wherein said human is no more than 70 years old.

39. The polyacrylamide hydrogel for use according to claim 35, wherein said human is female.

40. The polyacrylamide hydrogel for use according to claim 35, wherein said human is male.

41. The polyacrylamide hydrogel for use according to claim 35, wherein said human has a BMI of less than 25.

42. The polyacrylamide hydrogel for use according to claim 35, wherein said human has a BMI that is categorized as normal.

43. The polyacrylamide hydrogel for use according to claim 35, wherein an at least 50% reduction in WOMAC pain subscale is obtained in said human.

44. The polyacrylamide hydrogel for use according to claim 35, wherein an at least 12 points reduction in WOMAC pain subscale is obtained in said human.

45. The polyacrylamide hydrogel for use according to claim 35, wherein an at least 20 points reduction in WOMAC pain subscale is obtained in said human.

46. The polyacrylamide hydrogel for use according to claim 35, wherein about 40% of humans receiving the polyacrylamide hydrogel obtained an at least 50% reduction in WOMAC pain subscale in said human at week 26.

47. The polyacrylamide hydrogel for use according to claim 35, wherein about 50% of humans receiving the polyacrylamide hydrogel obtained an at least 12 points reduction in WOMAC pain subscale in said human at week 26.

48. The polyacrylamide hydrogel for use according to claim 35, wherein about 43% of humans receiving the polyacrylamide hydrogel obtained an at least 20 points reduction in WOMAC pain subscale in said human at week 26.

49. The polyacrylamide hydrogel for use according to claim 35, wherein the elasticity modulus of the polyacrylamide hydrogel is from about 10 Pa to about 200 Pa.

50. The polyacrylamide hydrogel for use according to claim 35, wherein the pH of the polyacrylamide hydrogel is more than about 5.

51. The polyacrylamide hydrogel for use according to claim 35, wherein the elasticity modulus of the polyacrylamide hydrogel is from 10 Pa to about 200 Pa and the pH of the polyacrylamide hydrogel is more than about 5.

52. The polyacrylamide hydrogel for use according to claim 35, wherein the polyacrylamide hydrogel is configured to be administered by injection under sterile conditions.

53. The polyacrylamide hydrogel for use according to claim 35, wherein 0.1-20 ml polyacrylamide hydrogel is administered by injection into the intraarticular cavity.

54. The polyacrylamide hydrogel for use according to claim 35, wherein the polyacrylamide hydrogel is administered by injection under sterile conditions into the intraarticular cavity at least once.

55. The polyacrylamide hydrogel for use according to claim 35, wherein about 6.0 ml of the polyacrylamide hydrogel is administered by injection under sterile conditions into the intraarticular cavity from one to ten times.

56. The polyacrylamide hydrogel for use according to claim 35, wherein about 3.0 ml of the polyacrylamide hydrogel is administered by injection under sterile conditions into the intraarticular cavity at least once or twice.

57. A method, comprising administering a polyacrylamide hydrogel to a human diagnosed with diagnosed with KL grade 2 and/or KL grade 3 osteoarthritis.

58. The method of claim 57, wherein the polyacrylamide hydrogel (i) has an elasticity modulus of from about 10 Pa to about 200 Pa, (ii) has a pH of more than about 5, or both (i) and (ii).

59. The method of claim 57, wherein the polyacrylamide hydrogel is administered by injection into an intraarticular cavity of the human.
